(19) United States
(12) Patent Application Publication
(10) Pub. No.: US 2003/0134824 A1
(43) Pub. Date: Jul. 17, 2003

(54) Beta-cyclodextrin dimers and phthalocyanines and uses thereof

(76) Inventors: Ronald Breslow, Englewood, NJ (US); Simon D.P. Baugh, Springfield, NJ (US)

Correspondence Address: Cooper & Dunham LLP, 1185 Avenue of the Americas, New York, NY, 10036, US

(21) Appl. No.: 10/054,585

(22) Filed: Nov. 12, 2001

(51) Int. Cl. A61K 31/724; C08B 37/16; A61K 31/555

(52) U.S. Cl. 514/58; 514/185; 536/46

(57) ABSTRACT

The invention provides β-cyclodextrin dimers and phthalocyanines which can be used in photodynamic therapy of cancer.

The invention disclosed herein was made with Government support under grant No. GM-18754 from the National Institutes of Health, U.S. Department of Health and Human Services, and CHE-97-12556 from the National Science Foundation. Accordingly, the U.S. Government has certain rights in this invention.

### BACKGROUND OF THE INVENTION

Throughout this application, various publications are referenced in parentheses. Full citations for these references may be found at the end of the specification immediately preceding the claims. The disclosures of these publications in their entireties are hereby incorporated by reference into this application to more fully describe the state of the art to which this invention pertains.

Photodynamic therapy of cancers uses a combination of light-activated drugs (photosensitizers) and laser light to create highly reactive forms of oxygen (singlet oxygen) that destroy tumor cells (Ali et al. 1999, Dougherty et al. 1998, Sternberg et al. 1998). Porphyrinoid dyes are photosensitizers which are widely used in photodynamic therapy. However, one major drawback of these hydrophobic photosensitizers is that they are not selective to tumor tissue because they are transported to every organ by blood lipoproteins of the blood stream (Moser et al., 1994). One way to prevent this is to attach the photosensitizer to cancer-specific antibodies and use cyclodextrin dimers to encapsulate the dye so that it cannot interact with lipoproteins (Ruebner et al., 1996, 1997).

Another strategy involves the use of β-cyclodextrin dimers having a cleavable linker between two β-cyclodextrin molecules to deliver the photosensitizer to the tumor site (U.S. Ser. No. 09/352,529, filed Jul. 13, 1999, now allowed; Ruebner et al. 1999). The β-cyclodextrin dimers can serve as hydrophilic carriers for photosensitizers, which can be administered to a subject with cancer. The β-cyclodextrin dimer can be cleaved by light, which can be selectively directed at the tumor site. The dye will then be released and be able to go into tumor cells. After the dimer is cleaved, the concentration of uncleaved β-cyclodextrin dimers at the tumor site will be reduced and more uncleaved β-cyclodextrin dimers will diffuse into the tumor site due to the concentration gradient. In this way, photosensitizer can be concentrated in the tumor without the use of a cancer-specific antibody.

The phthalocyanine described in U.S. Ser. No. 09/352,529, filed Jul. 13, 1999, now allowed, and in Ruebner et al. 1999 was a mixture of eight compounds. The present application discloses phthalocyanines with single well-defined structures, additional β-cyclodextrin dimers that can be used in photodynamic therapy, and phthalocyanines having characteristics that permit efficient cleavage of the β-cyclodextrin dimer-phthalocyanine complex.

### SUMMARY OF THE INVENTION

The invention provides a composition of matter comprising two β-cyclodextrin molecules and a cleavable linker joining each such β-cyclodextrin, wherein the cleavable linker comprises a carbon-carbon double bond substituted on both ends, wherein the cleavable linker is cleavable by singlet oxygen, and wherein the composition of matter is selected from the group consisting of:

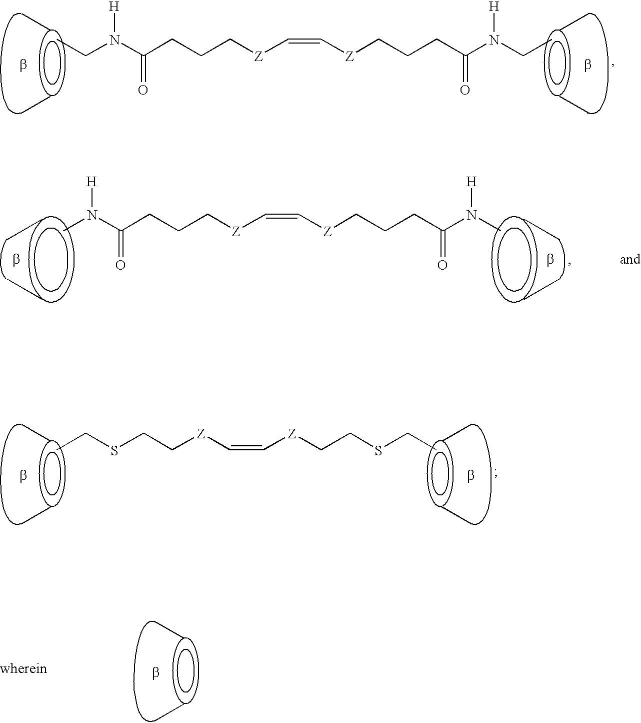

=beta-cyclodextrin; and

wherein Z is C₁-C₄ alkyl, NH, N(C₁-C₄ alkyl), O, or S.

The invention also provides a compound having the structure:

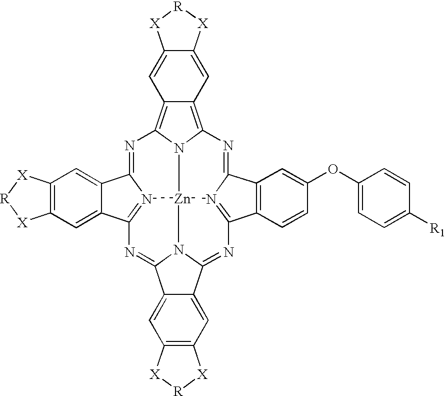

wherein X is C₁-C₄ alkyl, NH, N(C₁-C₄ alkyl) O, or S;

wherein R₁ is —CO₂H, —CO₂⁻, —N⁺(CH₃)₃, —SO₃H, or —SO₃⁻; and

wherein R is

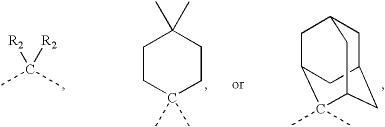

where the dashed lines indicate the attachments to X, and where R₂ is C₁-C₃ alkyl.

### BRIEF DESCRIPTION OF THE FIGURES

FIG. 1. β-Cyclodextrin dimers with photocleavable linkers as carriers for photosensitizers in the photodynamic therapy of cancers. The linker joining the β-cyclodextrin molecules 1 is cleavable by photoirradiation causing the release of the photosensitizer, which in the case illustrated is phthalocyanine 2.

FIG. 2. Synthesis of β-cyclodextrin dimers 17 and 18. Detailed description of synthesis is in text.

FIG. 3. Synthesis of β-cyclodextrin dimer 21. Detailed description of synthesis is in text.

FIG. 4. Determination of binding constant for β-cyclodextrin dimer 21 with BNS and with phthalocyanine 13. -x-: BNS titration into dimer 21 solution; -o-: ENS titration into phthalocyanine 13:dimer 21 (1:1) solution.

FIGS. 5a-5e. Conversion of β-cyclodextrindimer 21 to its cleavage product upon irradiation, with phthalocyanine 11, monitored by NMR. Reaction time: a, 30 minutes; b, 60 minutes; c, 90 minutes; d, 120 minutes; e, 150 minutes. Chemical shifts reported in parts per million (ppm) downfield of zero on the delta (5) scale α-axis).

### DETAILED DESCRIPTION OF THE INVENTION

The invention provides a composition of matter comprising two β-cyclodextrin molecules and a cleavable linker joining each such β-cyclodextrin, wherein the cleavable linker comprises a carbon-carbon double bond substituted on both ends, wherein the cleavable linker is cleavable by singlet oxygen, and wherein the composition of matter is selected from the group consisting of:

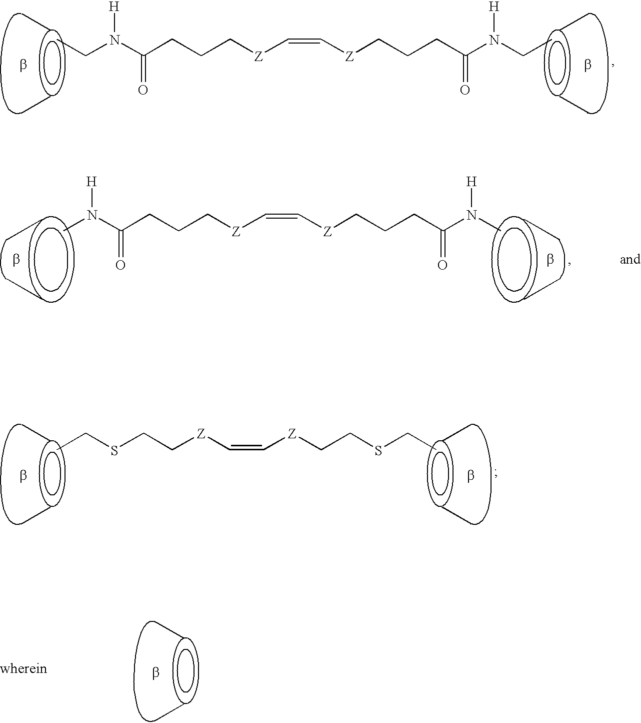

=beta-cyclodextrin; and

wherein Z is C₁-C₄ alkyl, NH, N(C₁-C₄ alkyl), O, or S.

In one embodiment of the composition of matter, Z is S.

In one embodiment, the composition of matter has the structure:

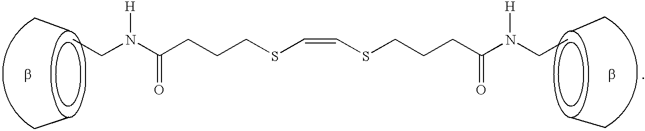

In one embodiment, the composition of matter has the structure:

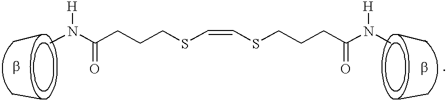

In one embodiment, the composition of matter has the structure:

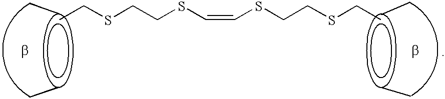

β-Cyclodextrin dimers with linkers of different lengths can be used to accommodate different size photosensitizers.

The invention provides a composition which comprises a hydrophilic matrix comprising the any of the compositions of matter disclosed herein and a photosensitizer encapsulated within the matrix. In different embodiments, the photosensitizer is a porphyrin, a phthalocyanine, a naphthalocyanine, a chlorine a pheophorbide, or a bacteriopheophorbide.

In one embodiment of the composition, the photosensitizer is a phthalocyanine. In one embodiment, the phthalocyanine has the structure:

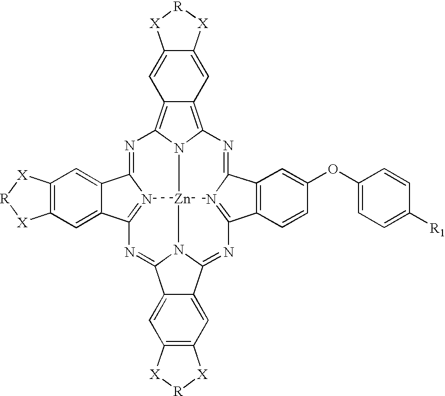

wherein X is C₁-C₄ alkyl, NH, N(C₁-C₄ alkyl), O, or S;

wherein R₁ is —CO₂H, —CO₂⁻, —N⁺(CH₃)₃, —SO₃H, or —SO₃⁻; and

wherein R is

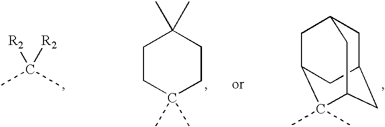

where the dashed lines indicate the attachments to X, and where R₂ is C₁-C₃ alkyl.

In one embodiment, X is O, and R₁ is —SO₃H.

In one embodiment, the phthalocyanine has the structure:

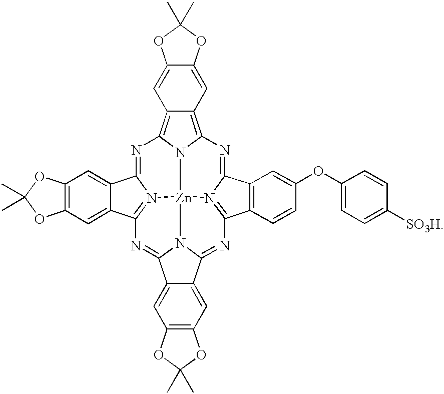

In one embodiment, the phthalocyanine has the structure:

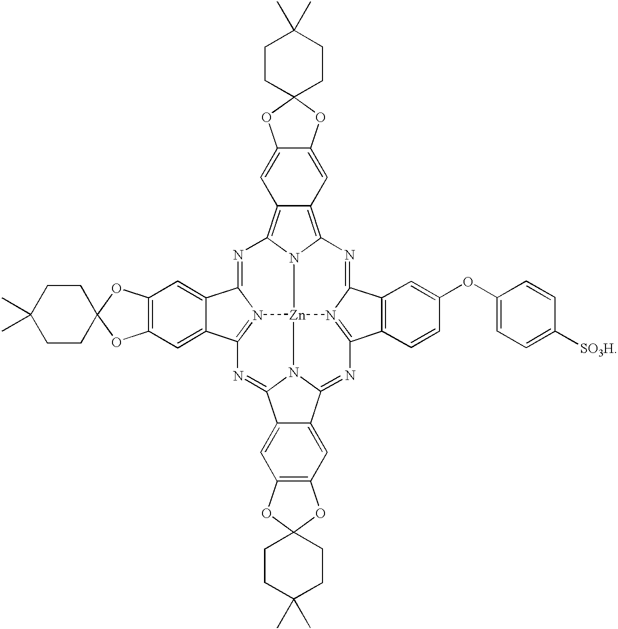

In one embodiment, the phthalocyanine has the structure:

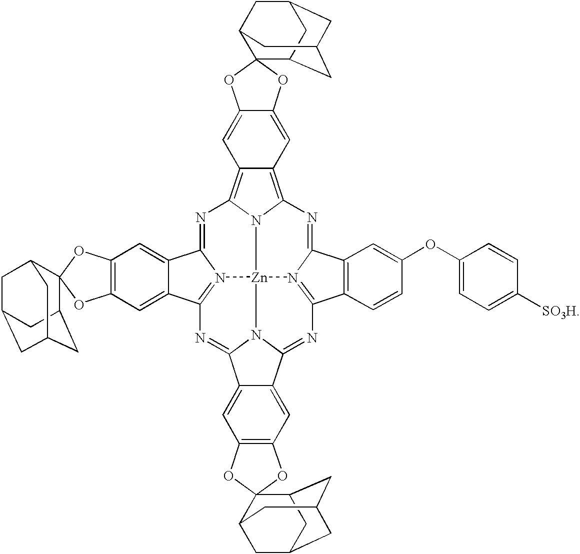

The invention provides a compound having the structure:

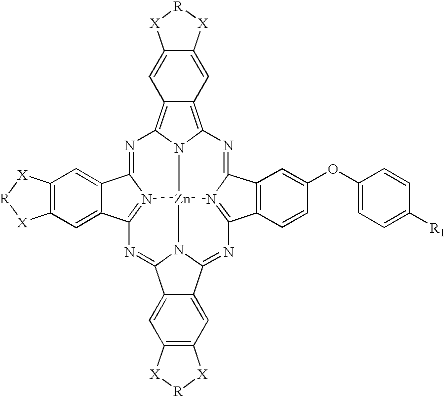

wherein X is C₁-C₄ alkyl, NH, N(C₁-C₄ alkyl), O, or S;

wherein R₁ is —CO₂H, —CO₂⁻; —N⁺(CH₃)₃, —SO₃H, or —SO₃⁻; and

wherein R is

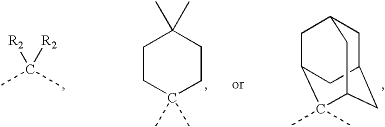

where the dashed lines indicate the attachments to X, and where R₂ is C₁-C₃ alkyl.

In one embodiment of the compound, X is O, and R₁ is —SO₃H.

In one embodiment, the compound has the structure:

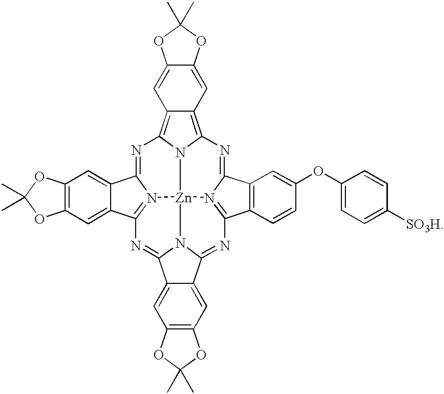

In one embodiment, the compound has the structure:

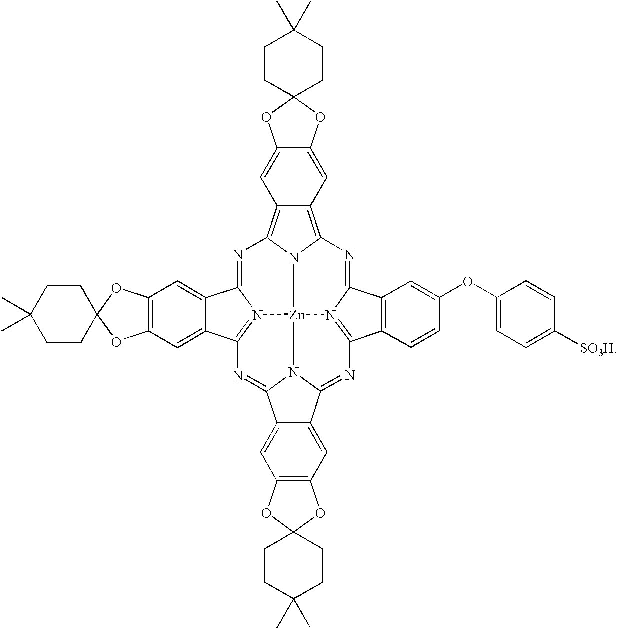

In one embodiment, the compound has the structure:

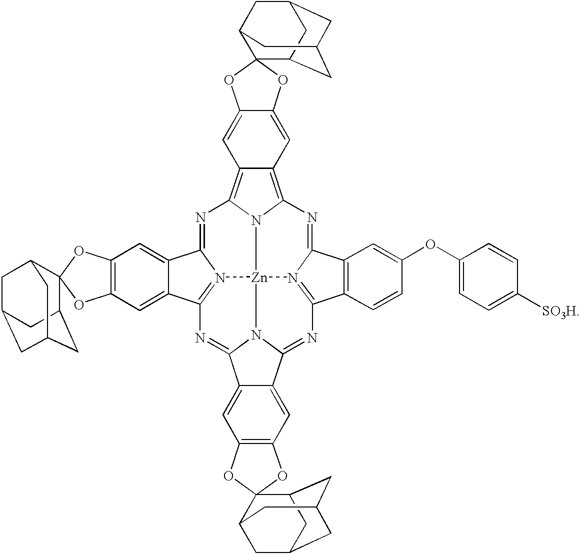

The invention provides a composition which comprises a hydrophilic matrix comprising:

i) any of the compounds disclosed herein encapsulated within the matrix, and

ii) a composition of matter comprising two β-cyclodextrin molecules and a cleavable linker joining each such β-cyclodextrin, wherein the cleavable linker comprises a carbon-carbon double bond substituted on one or both ends by an electron rich atom, and the cleavable linker is cleavable by singlet oxygen.

In different embodiments of the composition, the electron rich atom is sulfur, oxygen, or nitrogen.

In one embodiment of the composition, the composition of matter is selected from the group consisting of:

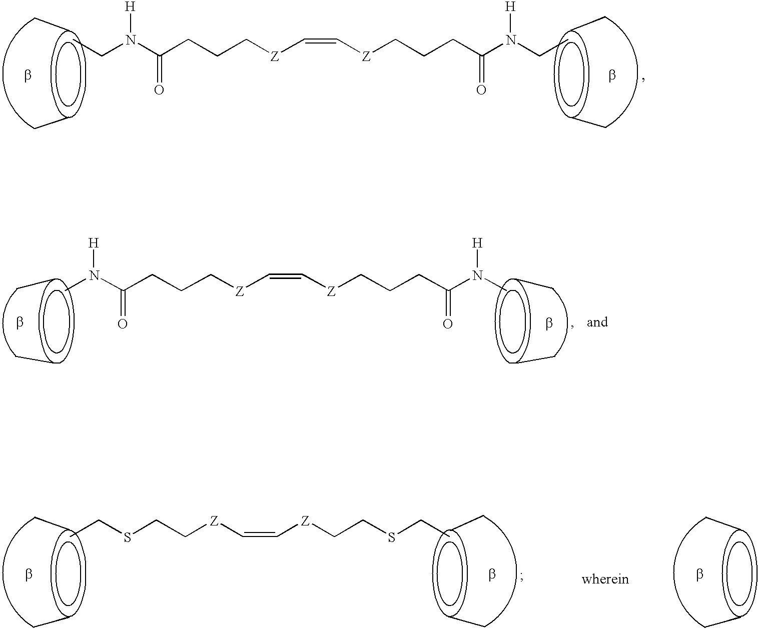

=beta-cyclodextrin; and

wherein Z is C₁-C₄ alkyl, NH, N(C₁-C₄ alkyl), O, or S.

In one embodiment, Z is S.

In one embodiment of the composition, the composition of matter has the structure:

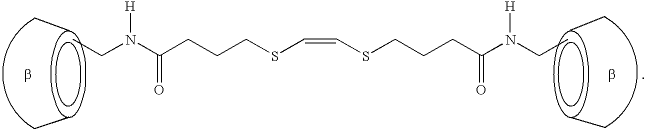

In one embodiment of the composition, the composition of matter has the structure:

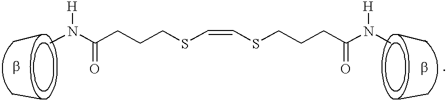

In one embodiment of the composition, the composition of matter has the structure:

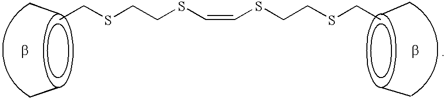

In one embodiment of any of the compositions disclosed herein, the cleavable linker is cleavable upon exposure to light of a wavelength appropriate for absorption by the photosensitizer. In one embodiment, the photosensitizer is released when the cleavable linker is cleaved.

The invention provides a method of killing a tumor cell which comprises contacting the tumor cell with any of the compositions disclosed herein and exposing the composition to light so as to cleave the cleavable linker and release the photosensitizer, wherein absorption of light by the photosensitizer excites the photosensitizer and the tumor cell is killed by singlet oxygen that is formed by energy transfer from the excited photosensitizer.

The invention provides a method of killing a tumor cell in a subject which comprises:

(a) administering any of the compositions disclosed herein to the subject;

(b) directing light at the tumor cell so as to expose the composition to light and cleave the cleavable linker thereby releasing the photosensitizer, wherein absorption of light by the photosensitizer excites the photosensitizer and generates singlet oxygen that is formed by energy transfer from the excited photosensitizer;

(c) allowing additional composition to diffuse to the tumor cell; and

(d) repeating steps (b) and (c) until sufficient singlet oxygen is generated to kill the tumor cell.

In one embodiment of any of the methods disclosed herein, the photosensitizer is concentrated at the tumor cell.

In one embodiment of any of the methods disclosed herein, a plurality of converging light beams is used to focus light on the tumor cell.

The compositions of matter and compounds disclosed herein may also be useful in applications other than the photodynamic therapy of cancer.

This invention will be better understood from the Experimental Details which follow. However, one skilled in the art will readily appreciate that the specific methods and results discussed are merely illustrative of the invention as described more fully in the claims which follow thereafter.

Experimental Details

The following Experimental Details are set forth to aid in an understanding of the invention, and are not intended, and should not be construed, to limit in any way the invention set forth in the claims which follow thereafter.

Background

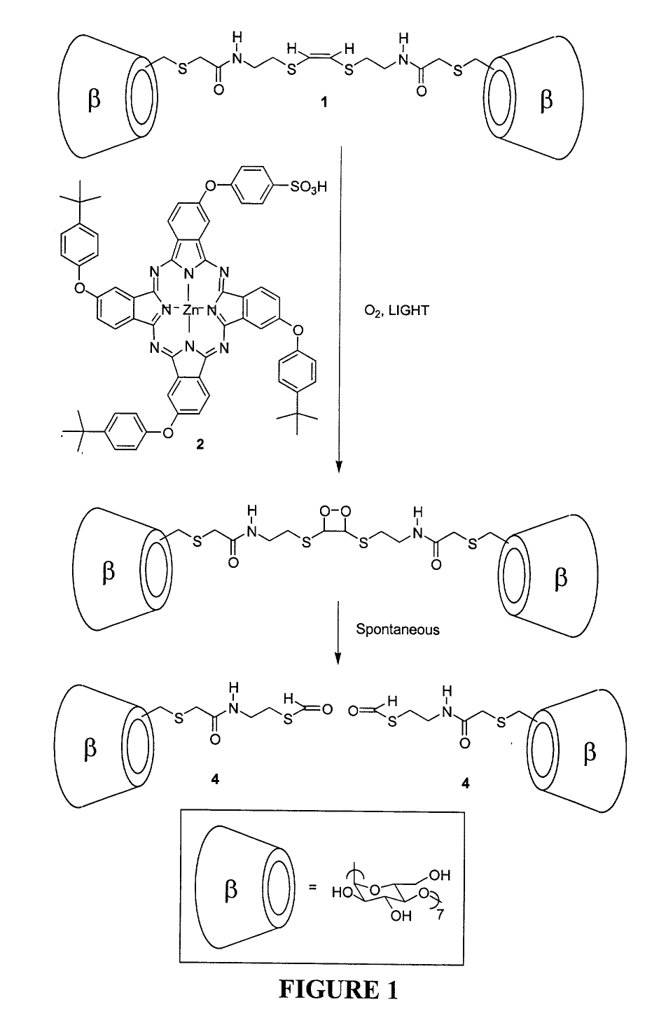

As previously disclosed (U.S. Ser. No. 09/352,529, filed Jul. 13, 1999, now allowed; Ruebner et al. 1999) and illustrated in FIG. 1, β-cyclodextrin dimer 1 and zinc phthalocyanine 2 formed a complex that is soluble in water. On irradiation of the complex in the presence of oxygen, the double bond of 1 is cleaved by singlet oxygen to form two moles of thioformate 4. Singlet oxygen adds to double bonds to form dioxetanes, which spontaneously fragment to generate carbonyl groups (Adam and Cliento 1983, Bartlett 1976, Clennan and Nagraba 1988, Foote 1971, Frimer 1979, Kearns 1971, Schaap and Zaklika 1979). The addition is particularly favorable for double bonds with electron donor substituents, as in 1. Since dimeric binding is stronger than the monomeric binding that occurs once the linker is cleaved, 4 then dissociates from 2. Furthermore, the chain of 4 almost certainly lowers the affinity of the cyclodextrin for the phthalocyanine by tucking back into the cyclodextrin cavity. An analog of 4 with a methyl group in place of the formyl group had an order of magnitude lower affinity for 4-tert-butylbenzoic acid than does simple β-cyclodextrin.

Phthalocyanine 2 is in fact a mixture of eight compounds, in which the substituents may be attached 3333 to the four phthalocyanine benzene rings (this is the structure shown, with the first number assigned as position 3 for the position of the sulfonate substituent on 2), 3233, 3323, 3332, 3223, 3322, 3232, or 3222 (Marcuccio et al. 1985). As described below, the present application discloses phthalocyanines with single well-defined structures. Phthalocyanines were chosen over porphyrins, because phthalocyanines are more stable to oxidation.

Synthesis of Zinc Phthalocyanines

An overview of the synthesis procedures is described in this section. Details are described in the “Detailed Synthesis” section at the end of “Experimental Details”.

Using a standard phthalocyanine synthetic procedure (Leznoff 1989), then incorporation of zinc, compounds 5 and 6 were prepared. Compound 5 is an analog of 2 with four equivalent substituents (it is a mixture of only four isomers since the 3333 isomer is the same as the 2222 isomer). Compound 6 is also an analog of 2 with a carboxyl instead of a sulfonate solubilizing group (again a mixture of eight isomers) (Kliesch et al. 1995).

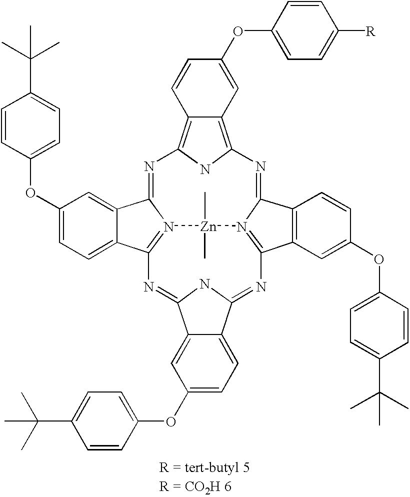

To achieve the symmetry that would give a single isomeric zinc phthalocyanine, phthalonitrile 7 was synthesized. This was made by brominating the catechol acetonide (Mitchell and Lai, 1979), then displacing the bromines with CuCN (the Rosenmund-von Braun reaction) (reviewed in Ellis and Romney-Alexander 1987). Conversion to the phthalocyanine with Li in pentanol, then treatment with zinc acetate, afforded compound 8. The elongated hydrophobically substituted 9 was synthesized from the corresponding phthalonitrile, synthesized by converting 3,4-dibromocatechol (Kohn 1951) to its ketal using 4,4-dimethylcyclohexanone (Meyer et al 1985), then displacing with CuCN. This was converted to the fully symmetrical zinc phthalocyanine 9 in a one-pot procedure with Li and zinc acetate (for an example of such transformation, see Lawrence and Whitten 1996).

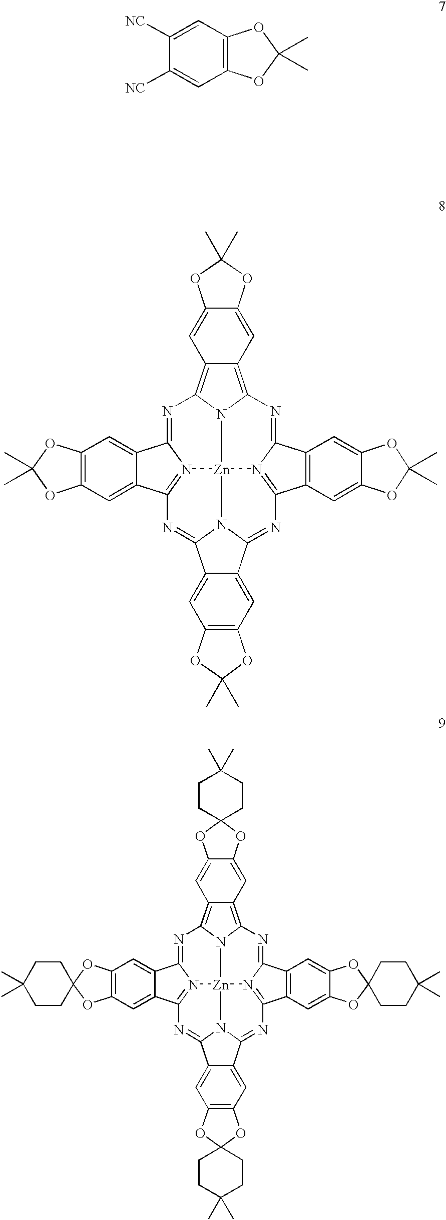

Adamantane derivatives bind well to β-cyclodextrin in water (Rekharsky and Inoue 1998), so zinc phthalocyanine 10 was also prepared from the ketal of catechol and 2-adamantanone (Takakis et al. 1992) by the bromination (Metz et al. 1984) and cyanide displacement sequence described herein, with the one-pot Li and Zn²⁺ method of cyclization. Two additional compounds were also prepared with one carboxyl solubilizing group, 11 and 12, by using a mixture of phthalonitriles and then separating the products. In the same manner the three monosulfonated analogs 13-15 were prepared. These compounds are all single isomers.

Finally, the commercially available zinc phthalocyanine 16 (Aldrich Chemical Company, Milwaukee), which is smaller than the other phthalocyanines, was also used in the studies described below.

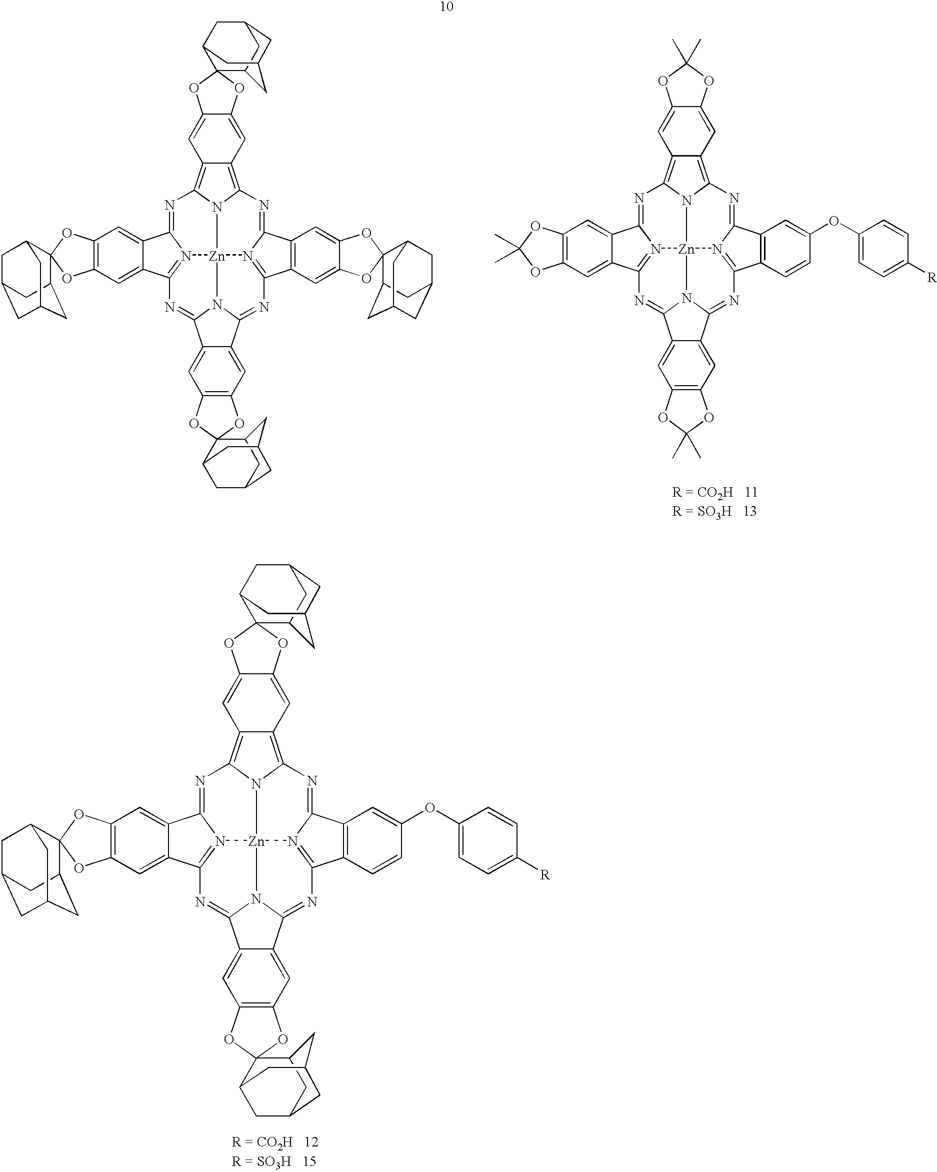

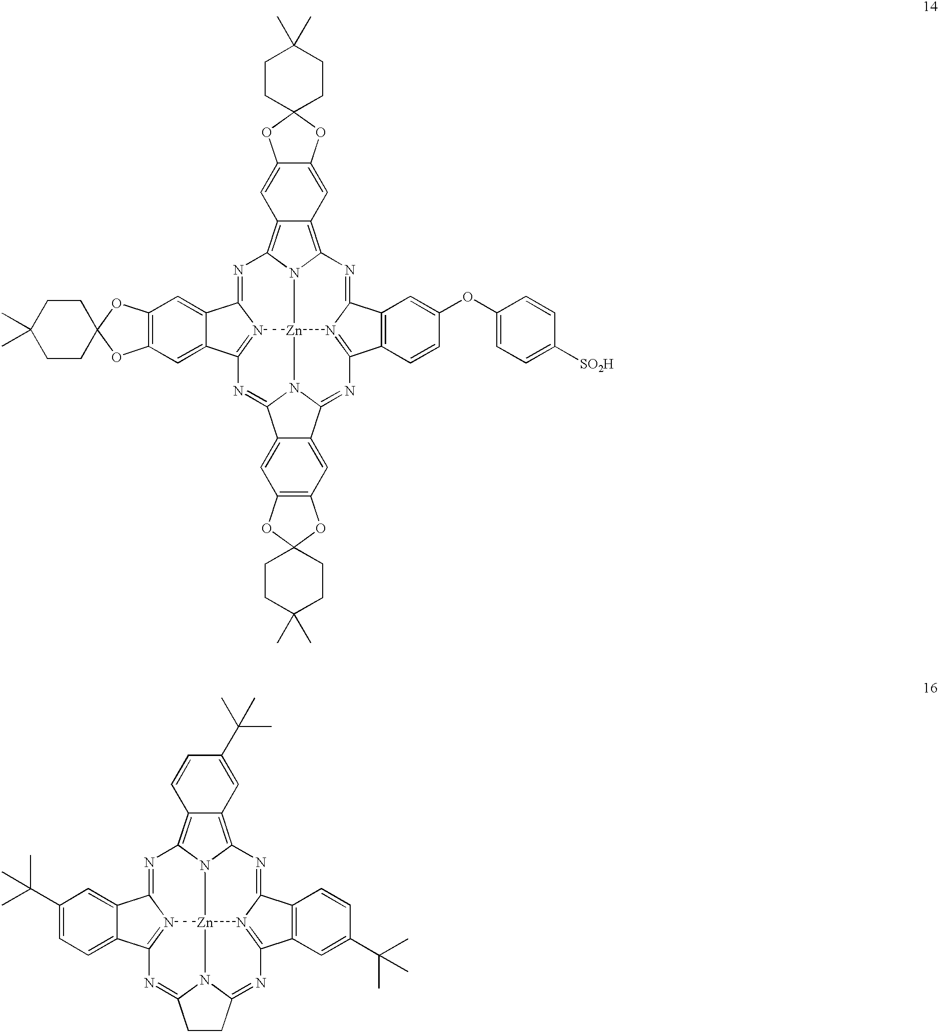

Synthesis of β-Cyclodextrin Dimers

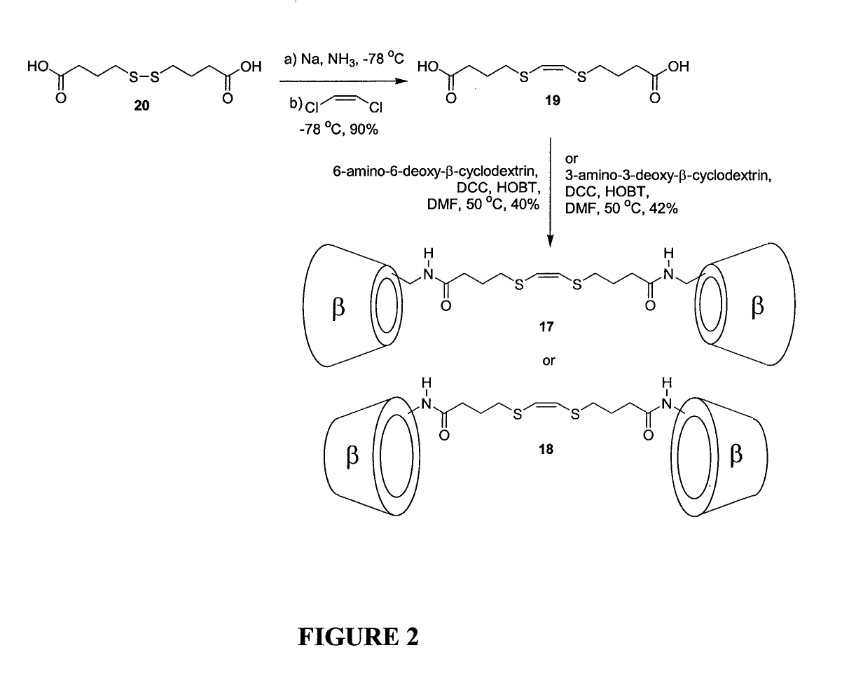

Dimer 1 was synthesized as described previously (U.S. Ser. No. 09/352,529, filed Jul. 13, 1999, now allowed; Ruebner et al. 1999). Dimers 17 and 18 were prepared as illustrated in FIG. 2 using linker 19 attached to the primary and secondary faces of β-cyclodextrin respectively. To make 19, disulfide 20 was reduced with sodium in ammonia, and then reacted with cis-1,2-dichloroethylene to afford diacid 19. This was then coupled with 6-deoxy-6-amino-β-cyclodextrin, using dicyclohexylcarbodiimide (DCC) and hydroxybenzotriazole (HOBt), to afford 17, and coupled with 3-deoxy-3-amino-β-cyclodextrin under the same conditions to afford 18.

The 3-deoxy-3-amino-β-cyclodextrin was prepared as previously described (Yuan et al. 1998) by preparing the 3-naphthalenesulfonate of β-cyclodextrin, closing it to the 2,3-alloepoxide with base and opening this with sodium azide. Reduction with triphenylphosphine then afforded 3-deoxy-3-amino-β-cyclodextrin, which was coupled with 19 to afford 18. The ¹H NMR spectrum of the azide showed that the attachment was on carbon 3 of the cyclodextrin, thus affording the product with overall retention of configuration. A small amount of the 2-azido compound was also formed, which was easily removed by chromatography.

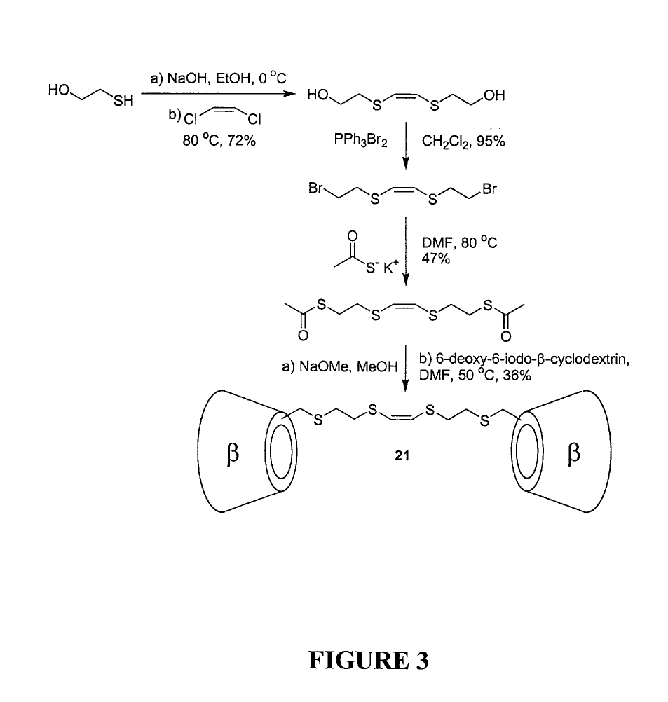

Finally, a β-cyclodextrin dimer with a shorter linker was synthesized, compound 21, as shown in FIG. 3. 2-Mercaptoethanol was coupled with cis-1,2-dichloroethylene, the hydroxyls then converted to bromides with triphenylphosphine dibromide, and the bromines replaced with potassium thioacetate. Then the acetate groups were removed with NaOMe, and the dithiolate was directly coupled with 6-deoxy-6-iodo-β-cyclodextrin to afford 21.

Binding Studies

Estimates of likely binding pairs were obtained by MacroModel simulations of the dimers and the phthalocyanines. The distances between the carbons of the two β-cyclodextrins to which the linkers are attached are: 1, 22 Å; 17, 20 Å; 18, 18 Å; 21, 16 Å. For the phthalocyanines, distances were measured from the methylated carbons across the ring for 5, 25 Å; 8, 17 Å and 9, 23 Å. For the adamantane derivative 10, the distance across the entire system, including all the adamantane atoms, was 23 Å. The MacroModel simulations also suggested that the alkene in the linker would be positioned directly above the metal center when the phthalocyanine is bound into the dimer. This would be expected to facilitate desired site-specific oxidation.

Binding constants for a few of the possible pairs were determined by a fluorescence competition method previously used for cyclodextrin dimers, competing the substrate of interest with 2-(p-tert-butylanilino)naphthalene-6-sulfonic acid 22, (termed BNS, Breslow et al. 1989).

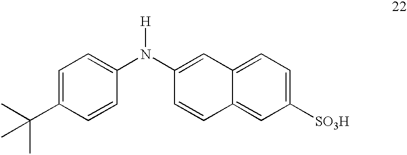

BNS is fluorescent when bound into a hydrophobic cavity such as that of a cyclodextrin, but only weakly fluorescent in water solution. Only the sulfonated phthalocyanines were soluble enough for this method.

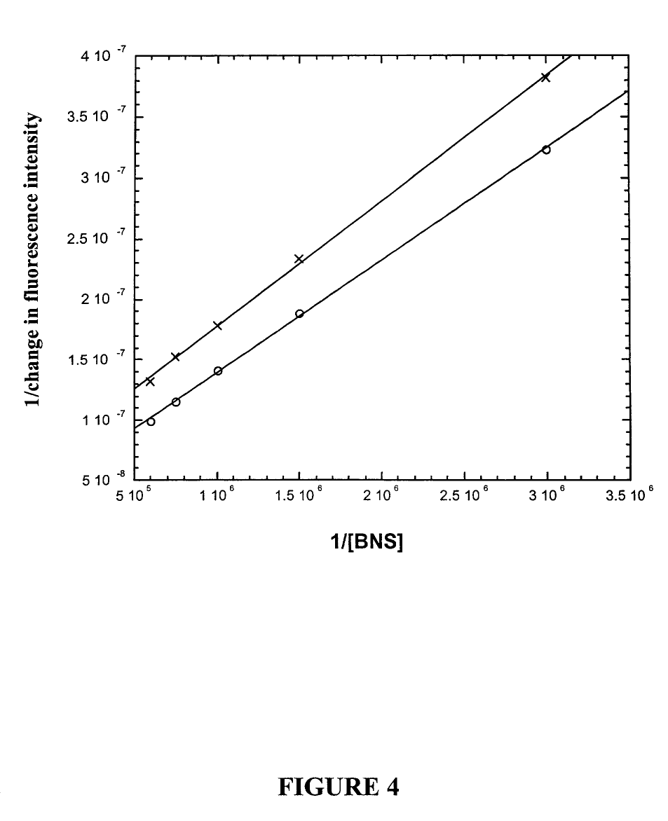

The binding constant of BNS 22 to each dimer was determined as previously (Breslow et al. 1989, Ruebner et al. 1999) by titrating BNS into a dimer solution in a fluorescence cell, then exciting this solution at 330 nm and measuring the fluorescent emission at 438 nm. The binding constant of phthalocyanine to dimer was measured by titration of BNS into a 1:1 mixture of dimer and phthalocyanine. The double reciprocal plot of change in fluorescent intensity and BNS concentration gives straight lines (for an example of such a plot, see FIG. 4). The straight lines found for each run show that the dimer and BNS were forming 1:1 complexes (Ruebner et al. 1996). The binding constant of BNS to each dimer is given by K=intercept/slope, and the binding constant of the phthalocyanine can be calculated as K_{I}=(K/K′−1)/[I], where K′ is the apparent binding constant of BNS to the dimer in presence of phthalocyanine, and [I] is the concentration of phthalocyanine (Ruebner et al. 1996).

The binding constants of BNS to the dimers used were as follows: 1, 1.93×10⁵ (reported 1.9×10⁵, Ruebner et al. 1999); 17, 1.07×10⁶; 18, 4.18×10⁵; 21, 7.01×10⁵. For the dimers with the phthalocyanines: 1/2, 2.00×10⁶ (reported 2×10⁶, Ruebner et al. 1999); 17/15, 6.05×10⁵; 18/14, 1.94×10⁶; 18/15, 1.76×10⁶; 21/13, 1.30×10⁶. All units in M⁻¹ at ca. 25° C.

As previously reported (Ruebner et al. 1999), there is some evidence that the linker chain in 1 is partly tucked into one cyclodextrin cavity in water solution, so these binding constants are somewhat diminished as a result. In the ¹H NMR spectrum of 1, the two vinyl protons are equivalent in DMSO solution, but non-equivalent in water. They become equivalent in water when hyodeoxycholic acid is added; this is known to bind strongly into β-cyclodextrin (Yang and Breslow 1997), and would thus be expected to displace the chain. Partial binding of the linker chain of 1 into one of the cyclodextrins in water leads to the non-equivalence, which apparently equilibrates slowly on the NMR time scale. This phenomenon was seen in the ¹H NMR spectra of all four of the cyclodextrin dimers described herein.

Photochemical Cleavage

All of the photocleavage reactions were carried out using the same apparatus, as described previously (U.S. Ser. No. 09/352,529, filed Jul. 13, 1999, now allowed; Ruebner et al. 1999). A halogen lamp (50 W) was set up with a 540 nm cut-off filter and a focusing lens. The NMR tube in which the reactions were run was placed in the most strongly focused area, and oxygen was bubbled continuously through the solution while the photocleavage reactions were run. To monitor the reactions, ¹H NMR's were taken at regular intervals, whereby the disappearance of the alkene peaks and the appearance of the single formyl peak could be observed.

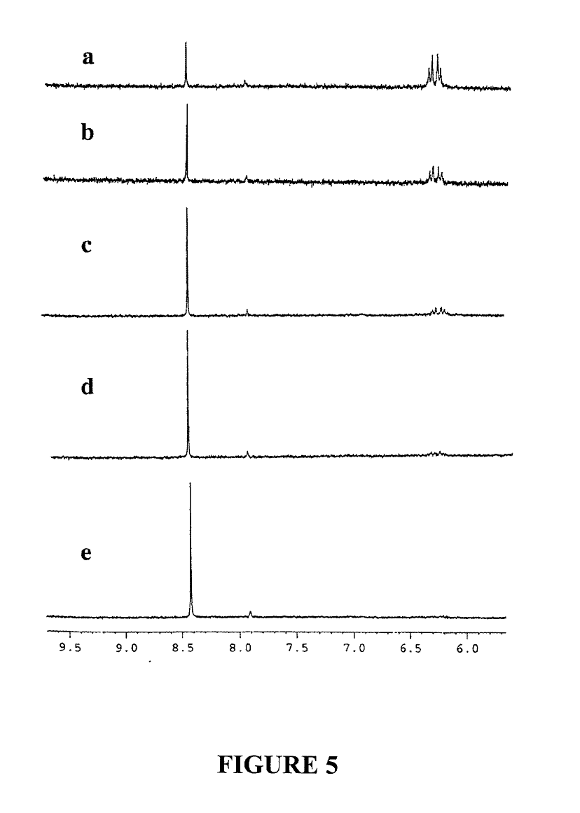

The dimers were at a concentration of 2.5 mM while the photosensitizers were at 0.14 mM in 5% DMSO-d₆ in D₂O. Typical ¹H NMR traces following the progress of a photocleavage reaction are shown in FIG. 5. Here dimer 21 was cleaved using phthalocyanine 11. It can clearly be seen that the amount of alkene (doublet at ˜6.2 ppm) decreases and the formyl peak (singlet at ˜8.4 ppm) appears as the reaction progresses, and that no other peaks are seen in this area.

The plots of percent cleavage vs. time were all linear, indicating that the phthalocyanines remain active during the reactions and the light intensity is essentially constant. Furthermore, the NMR tubes were repeatedly removed from the apparatus, wrapped in aluminum foil, and taken to the NMR machine, then returned to the apparatus. The consistency of data indicates that the light flux was consistent throughout the runs. Also, repeats of the 21/13 experiment more than three weeks apart gave values of 5.3 and 5.4 min for 50% cleavage, showing that the photolysis apparatus is stable.

Comparison was made of the relative times needed for 50% cleavage of the dimers by various bound phthalocyanines under the conditions above, at ambient temperature (25° C.). The data are listed in Table 1.

TABLE 1
Times for 50% cleavage of the dimers (min)

| | Phthalocyanine | | | | | | | | | |
|---|---|---|---|---|---|---|---|---|---|---|
| | 2 | 5 | 6 | 8 | 11 | 12 | 13 | 14 | 15 | 16 |
| Dimer 1 | 7 | | | | | 60 | | | 6 | |
| Dimer 17 | | 80 | 40 | | | | | | | |
| Dimer 18 | | | 55 | | | | | 6 | | |
| Dimer 21 | | | 85 | 180 | 55 | | 5.3; | | | 22% in |
| | | | | | | | 5.4 | | | 180 min |

The dimers were at a concentration of 2.5 mM while the photosensitizers were at 0.14 mM in 5% DMSO-d₆ in D₂O.

As the data indicate, there is considerable variation in the effectiveness of the photolytic cleavage process. Dimer 1 is rapidly cleaved by the previously made sulfonate sensitizer 2, a mixture of eight isomers, and also by the well-defined adamantyl sulfonate sensitizer 15, but not as rapidly by the corresponding carboxylate 12. Dimer 17 was not rapidly cleaved by either 6, the carboxylate version of 2, or by 5, the version of 2 with no solubilizing group.

The importance of a solubilizing group is clear. Sensitizers 5, 8, and 16 have no sulfonate or carboxylate group, and all their cleavage reactions are slow. Sensitizers 6, 11, and 12 have only carboxylate solubilizing groups, and are not as effective as the sulfonates in 2, 13, 14, and 15. As mentioned above, the high reproducibility of the data for the 21/13 cleavage reaction, data collected more than three weeks apart, indicates that these data are reliable. Since the compounds were apparently in solution during these runs, it is likely that the better solubilizing groups produce a faster off rate once the linker is cleaved, permitting the turnovers that are involved in these processes with their excess of dimer over photosensitizer.

Good agreement of the experimental data with the MacroModel information can also be seen here. According to the MacroModel calculations, phthalocyanine 6 would be expected to bind most effectively with dimer 17 and least effectively with dimer 21. This agrees very well with the experimental data, where the times for 50% cleavage of dimers 17, 18 and 21 by phthalocyanine 6 are 40, 55 and 85 minutes respectively.

Control reactions were performed. When the oxygen was replaced by argon there was no cleavage, nor any in the absence of the sensitizer under the normal conditions. When the sensitizer was replaced by methylene blue, dimer 1 was cleaved to product 4 with its ¹H NMR peak at 8.4 ppm for the formyl group, but additional peaks were also seen at 10.2 and 7.8 ppm. Apparently singlet oxygen generated this way, rather than in complex 3, is able to attack the cyclodextrin ring also. As pointed out previously (U.S. Ser. No. 09/352,529, filed Jul. 13, 1999, now allowed; Ruebner et al. 1999), this indicates that the singlet oxygen generated in the complex 3 is selectively taken up by the nearby olefin linkage of 1. After dimer 21 had been completely cleaved by sensitizer 13, in ca. 10 minutes, irradiation was continued for an additional 20 minutes but produced no further change in the ¹H NMR of the solution. In accordance with this result, when the cyclodextrin dimer was replaced by β-cyclodextrin and the reaction was run under normal conditions, no oxidation products were observed.

The cleavage of dimers diminishes their affinity for the sensitizers, and not just because the chelate effect is gone. Examination was made of the binding of compound 23, which mimics the cleavage product 4, except with a methyl group replacing the somewhat hydrolytically labile formyl group.

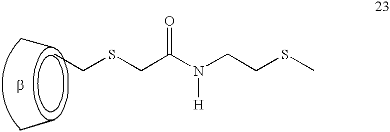

With 4-tert-butylbenzoic acid, β-cyclodextrin has a binding constant of 1.7×10⁴ M⁻, while 23 had a binding constant of only 1.8×10³ M⁻¹, an order of magnitude less. This difference can be ascribed to the competitive binding of the chain in 23 into the cyclodextrin cavity. It is proposed that the same interaction occurs with cleavage product 4 and related cleavage products from the other dimers.

Concentration of the sensitizer complex into a light beam was achieved using an experiment described previously (Ruenber et al. 1999) in which a tube was shielded with aluminum foil so that only a small section could be irradiated through a window in the foil. Phthalocyanine 13 and dimer 21 were made up in a 1:1 solution in D₂O. A small amount of 13 was insoluble, and removed. The solution was then irradiated through the window, and after 40 hours sensitizer 13 had precipitated solely in the window, and the ¹H NMR of the solution indicated that all the dimer 21 had been cleaved; all the vinyl protons were gone, replaced by formyl protons of the monomer. Thus, as expected, the dissolved components do diffuse into the light beam, where they undergo the cleavage reaction.

Characterization of the Dimers and the Phthalocyanines.

Dimer 1: ¹H NMR (300 MHz, DMSO-d₆) δ 8.09 (t, 2H), 6.22 (s, 2H), 5.90-5.60 (m, 28H), 4.95-4.75 (m, 14H), 4.55-4.40 (m, 12H), 3.90-3.45 (m, 56H), 2.77 (t, 4H); MS (FAB): 2560 (M+2⁺, 10).

Dimer 17: ¹H NMR (300 MHz, DMSO-d₆) δ 7.68 (bs, 2H), 6.16 (s, 2H), 5.80-5.67 (m, 28H), 4.81 (m, 14H), 4.47 (m, 14H), 3.62-3.32 (m, 84H), 2.72-2.66 (m, 4H) 2.21-2.10 (m, 4H), 1.84-1.66 (m, 4H); MS (MALDI) m/z (%) 2517 (M+1+Na⁺, 5).

Dimer 18: ¹H NMR (300 MHz, DMSO-d₆) δ 7.90 (bs, 2H), 6.20 (s, 2H), 5.99 (d, J=6.1, 2H), 5.79-5.61 (m, 26H), 4.82-4.75 (m, 14H), 4.50-4.45 (m, 14H), 3.62-3.32 (m, 84H), 2.73 (m, 4H), 2.26-2.23 (m, 4H), 1.82-1.79 (m, 4H); MS (MALDI) m/z (%) 2516 (M+Na⁺, 15).

Dimer 21: TLC R_{f} 0.13 7:7:5 i-PrOH:Ethyl Acetate:Water; ¹H NMR (300 MHz, DMSO-d₆) δ 6.24 (s, 2H), 5.78-5.67 (m, 28H), 4.84 (s, 14H), 4.49-4.43 (m, 12H), 3.87-3.30 (m, 80H), 3.05-2.64 (m, 12H).

Zinc phthalocyanine 2: TLC R_{f} 0.24 20% MeOH (10% NH₃):CHCl₃; ¹H NMR (300 MHz, CDCl₃) δ 9.45-9.30 (m, 4H), 8.92-8.73 (m, 4H) 7.89-7.81 (m, 3H), 7.70-7.61 (m, 8H) 7.45-7.35 (m, 8H), 1.47-1.41 (m, 27H)); MS (FAB) m/z (%) 1193 (M+H⁺, 5); λₘₐₓ (nm) 678.

Zinc phthalocyanine 5: TLC R_{f} 0.67 10% MeOH (10% NH₃):CHCl₃; ¹H NMR (300 MHz, CDCl₃) δ 8.72-7.78 (m, 8H), 7.60-7.51 (m, 4H), 7.38-7.07 (m, 16H), 1.55-1.42 (m, 36H); MS (FAB) m/z (%) 1170 (M+H⁺, 2); λₘₐₓ (nm) 680.

Zinc phthalocyanine 6: ¹H NMR (300 MHz, CDCl₃) δ 9.22-8.60 (m, 8H), 8.25-7.35 (m, 20H), 1.46-1.35 (m, 27H); MS (FAB) m/z (%) 1157 (M+H⁺, 5).

Zinc phthalocyanine 8: ¹H NMR (300 MHz, CDCl₃) δ 7.15-7.05 (m, 8H), 1.81-1.73 (m, 24H); MS (APCI) m/z (%) 864 (M⁺, 4); λₘₐₓ (nm) 667.

Zinc phthalocyanine 9: ¹H NMR (300 MHz, CDCl₃) δ 8.15 (bs, 8H), 2.35 (bs, 16H), 1.88 (bs, 16H), 1.24 (s, 26H); MS (FAB) m/z (%) 1137 (M+H⁺, 6), 1136 (M⁺, 5); λₘₐₓ (nm) 669.

Zinc phthalocyanine 10: TLC R_{f} 0.80 10% MeOH (10% NH₃):CHCl₃; 1H NMR (300 MHz, CDCl₃) δ 8.60 (bs, 8H), 1.61-2.67 (m, 56H); MS (FAB) m/z (%) 1235 (M+H+, 5); λₘₐₓ (nm) 668.

Zinc phthalocyanine 11: TLC R_{f} 0.24 20% MeOH (10% NH₃):CHCl₃; 1H NMR (300 MHz, DMSO-d₆) δ 8.71 (m, 4H), 8.13-8.01 (m, 5H), 7.71-7.23 (m, 4H), 5.76 (s, 1H) 1.96 (s, 18H); MS (FAB) m/z (%) 930 (M+H⁺, 2) 929 (M⁺, 2); λₘₐₓ (nm) 666.

Zinc phthalocyanine 12: TLC R_{f} 0.25 20% MeOH (10% NH₃):CHCl₃; ¹H NMR (300 MHz, DMSO-d₆) δ 9.38-9.33 (m, 1H), 8.94-8.90 (m, 1H), 8.58-8.42 (m, 4H), 8.16 (d, J=8.4, 2H), 8.04-7.92 (m, 1H), 7.58 (s, 1H), 7.49 (d, J=8.7, 2H), 7.27 (s, 1H), 2.33-1.73 (m, 42H); MS (FAB) m/z (%) 1205 (M⁺, 0.5); λₘₐₓ (nm) 667.

Zinc phthalocyanine 13: TLC R_{f} 0.20 20% MeOH (10% NH₃):CHCl₃; ¹H NMR (300 MHz, DMSO-d₆) δ 9.42-9.36 (m, 1H), 8.72-8.50 (m, 4H), 8.41-8.21 (m, 1H), 7.78 (d, J=8.7, 2H), 7.64-7.53 (m, 1H), 7.32 (d, J=9.0, 2H), 7.25 (s, 1H), 7.11 (s, 1H), 1.98-1.95 (m, 6H), 1.71-1.65 (m, 12H); MS (FAB) m/z (%) 966 (M+H⁺, 1.5), 965 (M⁺, 1.5); λₘₐₓ (nm) 665.

Zinc phthalocyanine 14: TLC R_{f} 0.21 20% MeOH (10% NH₃):CHCl₃; ¹H NMR (300 MHz, DMSO-d₆) δ 9.38-9.28 (m, 1H), 8.89-8.82 (m, 1H), 8.67-8.49 (m, 4H), 7.82 (d, J=7.5, 2H), 7.37 (d, J=8.1, 2H), 7.25 (d, J=7.2, 1H), 7.03 (s, 1H), 6.87 (d, J=7.5, 1H), 2.27 (bs, 4H), 1.96 (bs, 4H), 1.74 (bs, 4H), 1.49 (bs, 4H), 1.22-0.74 (m, 26H); MS (FAB) m/z (%) 1171 (M⁺, 30); λₘₐₓ (nm) 666.

Zinc phthalocyanine 15: TLC R_{f} 0.25 20% MeOH (10% NH₃):CHCl₃; ¹H NMR (300 MHz, DMSO-d₆) δ 9.378 (bs, 2H), 8.93-8.85 (m, 1H), 8.70-8.52 (m, 3H), 7.80 (d, J=7.5, 2H), 7.54 (s, 1H), 7.50 (d, J=9.9, 1H), 7.37 (d, J=7.5, 2H), 7.05 (s, 1H), 2.26-1.72 (m, 42H) MS (FAB) m/z (%) 1243 (M⁺, 10); λₘₐₓ (nm) 667.

Details of Binding Studies

Monomer 23 with p-tert-butylbenzoic Acid

β-cyclodextrin (CD) monomer 23 (6.4 mg, 0.005 mmol) was dissolved in 0.20 M pH 9.0 Na₂CO₃/NaHCO₃ buffer (5.00 mL) to make a 1.0 mM solution. p-tert-Butylbenzoic acid (11.0 mg, 0.062 mmol) was dissolved in 6.16 mL of the same buffer to make a 10.0 mM solution. Both solutions were degassed under reduced pressure with a sonicator for 5 minutes immediately prior to the binding study. The β-CD monomer 23 solution (2.50 mL) was put into the sample cell compartment of an Omega microcalorimeter, whereas the p-tert-butyl-benzoic acid was loaded in a 250 μL syringe and then assembled onto the calorimeter. The system was equilibrated until RMS error was less than 5×10⁻³ with the syringe spinning at 400 rpm. The p-tert-butylbenzoic acid solution was then injected into the cell in 25 injections (10 μL, 7 seconds per injection). The time interval between injections was set to be 4 minutes. Injection data were automatically collected by the computer, and the data was analyzed by ORIGIN software with the single-binding-site model. Two trials were performed.

| | Trial 1 | Trial 2 |
|---|---|---|
| Binding constant: | 1.8 ± 0.2 × 10³ M⁻¹ | 1.8 ± 0.2 × 10³ M⁻¹ |
| Binding ratio: | 0.91 ± 0.07 | 0.99 ± 0.07 |

Binding of β-cyclodextrin to p-tert-butylbenzoic Acid

β-Cyclodextrin (4.0 mg, 0.0035 mmol) was dissolved in 0.20 M pH 9.0 Na₂CO₃/NaHCO₃ buffer (3.53 mL) to make a 1.0 mM solution. p-tert-butyl-benzoic acid (10.0 mg, 0.056 mmol) was dissolved in 5.60 mL of the same buffer to make a 10.0 mM solution. Both solutions were degassed under reduced pressure with a sonicator for 5 minutes immediately prior to the binding study. The β-Cyclodextrin solution (2.50 mL) was put into the sample cell compartment of the microcalorimeter, whereas the p-tert-butyl-benzoic acid was loaded in a 250 μL syringe and then assembled onto the calorimeter. The whole setup was equilibrated until RMS error was less than 5×10⁻³ with the syringe spinnning at 400 rpm. The p-tert-butyl-benzoic acid solution was then injected into the cell in 25 injections (10 μL, 7 seconds per injection). The time interval between injections was set to be 4 minutes. Injection data were automatically collected by the computer, and the data was analyzed by ORIGIN software with the single-binding-site model. Binding constant: 1.7±0.2×10⁴ M⁻¹.

Representative Binding Study of a Phthalocyanine to a Cyclodextrin Dimer.

Three solutions were made:

i) β-cyclodextrin dimer 21 (0.20 mg, 8.18×10⁻⁸ mol) was dissolved in degassed water (250 mL).

ii) BNS 22 (0.14 mg, 3.94×10⁻⁷ mol) was dissolved in degassed water (4.00 mL).

iii) β-cyclodextrin dimer 21 (0.40 mg, 1.64×10⁻⁷ mol) and phthalocyanine 13 (1.64×10⁻⁷ mol) were dissolved in a mixture of methanol (1 mL) and water (0.1 mL). This mixture was stirred in the dark for 1 h. The solution was then concentrated under vacuum and then placed under high-vacuum for 18 h. The resulting material was dissolved in degassed water (500 mL).

The binding constant for BNS to cyclodextrin dimer 21 was determined by the fluorescence emission method. Cyclodextrin dimer 21 solution (3.00 mL) was added to a fluorescence cell and was excited at 330 nm and the emission at 418 nm was measured. Five additions of BNS solution (10 μL) were made, with a measurement being taken after each one. The area under the peak between 400 and 500 nm was measured for each addition. The experiment was run in duplicate.

The binding constant for phthalocyanine 13 to dimer 21 was determined by the fluorescence emission method. 1:1 Dimer: phthalocyanine solution (3.00 mL) was added to a fluorescence cell. This solution was excited at 330 nm and the emission at 418 nm was measured. Five additions of BNS solution (10 μL) were made, with a measurement being taken after each one. The experiment was run in duplicate. The data are plotted in FIG. 4.

Representative Photocleavage Procedure

To a solution of β-cyclodextrin dimer 17 (5.9 mg, 2.35×10⁻⁶ mol) and potassium carbonate (2 mg) in D₂O (1.00 mL) was added a 3 mM solution of phthalocyanine 6 (0.17 mg, 1.50×10⁻⁷ mol) in DMSO-d₆ (50 μL). This solution was transferred to an NMR tube and was irradiated with a halogen lamp (50 W) with a cut-off filter to exclude wavelengths below 540 nm. During irradiation, oxygen was bubbled through the solution. The reaction was monitored by ¹H NMR, by observance of the disappearance of the alkene peaks and the appearance of the formyl peak. Whenever the ¹H NMR was taken, the NMR tube was completely shielded from light using an aluminum foil cover during transportation to the NMR room. The results of a typical run are shown in FIG. 5.

Representative Precipitation Experiment Procedure

β-Cyclodextrin dimer 21 (2.4 mg, 1.00×10⁻⁶ mol), potassium carbonate (0.5 mg) and phthalocyanine 13 (0.97 mg, 1.00×10⁻⁶ mol) were dissolved in a mixture of methanol (1 mL) and D₂O (0.1 mL). The mixture was stirred in the dark for 1 h. The solvents were removed under vacuum and the resulting solid was placed under high vacuum for 18 h. The residue was treated with D₂O (1.00 mL) and stirred in the dark for 2 h, during which time everything appeared to have dissolved. The solution was then filtered through a cotton wool plug and transferred to an NMR tube. The tube was covered in aluminum foil except for an area approximately 0.5 cm wide, which was left uncovered, through which the solution could be seen. Oxygen was bubbled through the solution for 5 min while the solution was kept in the dark. The solution was then placed on its side and was irradiated with a halogen lamp (50 W) with a cut-off filter to exclude wavelengths below 540 nm. The solution was re-saturated with oxygen, in the same manner as above, after 18 h.

Detailed Synthesis

In the Detailed Synthesis section only, the numbering of structures differs from that in the remainder of the application since many more numbered structures are included here. The corresponding numbers are as follows: remainder of application:(Detailed Synthesis)—1:(2), 2:(1), 5:(14) 6:(17), 7:(22) 8:(24), 9:(29), 10:(34), 11:(35), 12:(36), 13:(39) 14:(40), 15:(41), 16:(42), 17:(43), 18:(44), 19:(45), 20:(46), 21:(67), 22:(74), 23:(71).

Solvents, drying reagents and inorganic salts were obtained from Aldrich Chemical Company or Fisher Scientific Company and used without further purification unless otherwise specified. β-cyclodextrin was obtained from American Maize Company. THF and CH₂Cl₂ were dried by distillation under argon from Na.Benzophenone and calcium hydride respectively. Anhydrous DMF was obtained from Aldrich in SureSeal™ bottles. Argon was obtained from Matheson.

¹H NMR spectra were recorded on Bruker DMX 300, 400 or 500 MHz or Varian VXR 400 MHz instruments with the residue solvent peaks as the reference signal. TMS (tetramethyl silane) was used as internal reference for the measurements in CDCl₃. ¹³C spectra were recorded on Varian VXR 300 MHz or Bruker DMX 300 MHz instruments. All chemical shifts were reported in parts per million (ppm) downfield of zero on the delta (δ) scale.

Mass spectra were recorded on a Nermag R-10-10 spectrometer for CI and EI spectra, or a Jeol JMS-DX-303 HF instrument for FAB spectra. Infrared spectra were recorded on a Perkin-Elmer 1600 Fourier Transform spectrometer. Ultraviolet/Visible (UV-vis) spectra were recorded on a Varian CARY 1E spectrometer. Microcalorimetic titrations were performed on an OMEGA calorimeter. Melting points were determined using MelTemp capillary melting point apparatus and were uncorrected. Cyclodextrin products were dried by a VirTis Sentry Lyophiliser.

Analytical thin layer chromatography (TLC) was performed on 0.25 mm precoated silica gel plates with 254 nm fluorescence indicator from EM science. Compounds were visualised under UV light or by TLC staining solutions. All cyclodextrin compounds were stained with anisaldehyde stain (a solution of p-anisaldehyde (9.2 mL), glacial acetic acid (3.7 mL) and concentrated H₂SO₄ (12.5 mL) in 190 proof ethanol (340 mL), and then heated until the blue-gray spots appear.

Silica gel column chromatography was performed with 230-400 mesh silica from EM science. All reverse phase column chromatography was performed using homemade C-18 reverse phase silica gel. The compounds containing β-cyclodextrins were dissolved in water and loaded onto the column, then eluted with the linear gradient of solvent systems described.

All reactions were performed under an atmosphere of argon unless otherwise specified.

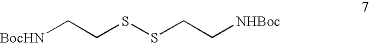

Cystamine (4.00 g, 17.7 mmol) was dissolved in a mixed solvent system of dioxane/water (1:1) (30 mL), followed by the addition of sodium hydroxide (1.40 g, 35.4 mmol). Boc₂O (8.20 g, 38.8 mmol) was added after the solution was cooled to 0° C. The solution was warmed to 25° C. and stirred for 30 min. After a few minutes a precipitate appeared. The solvent was removed under reduced pressure and the residue was dissolved in ethyl acetate. The insoluble parts (salts) were removed by filtration, and the filtrate was washed with 1 M HCl (10 mL), water (10 mL), and 1 M NaOH (10 mL), then dried (MgSO₄). Concentration of the solution gave [2-(2-tert-butyoxycarbonylamino-ethyldisulfanyl)-ethyl]carbamic acid tert-butyl ester 7 (6.40 g, 86%) as a white solid. Mp 105° C.; ¹H NMR (300 MHz, DMSO-d₆) δ6.95 (t, J=5.4, 2H), 3.16-3.23 (m, 4H), 2.51 (t, J=7.0, 4H), 1.37 (s, 18H); ¹³C NMR δ 155.5, 77.7, 37.6, 28.2; MS (APCI) m/z (%) 353 (M+H⁺, 20).

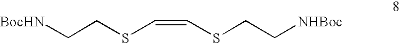

Diamide 7 (2.00 g, 5.60 mmol) was placed in a small 3-necked-flask which was evacuated and backfilled with argon 3 times. Ammonia (30 mL) was condensed into the flask using a cold trap. Pieces of sodium metal were added to the flask until the blue colour remained. The solution was stirred for 30 minutes, and more sodium was added if the blue colour disappeared. A minimum amount of solid ammonium chloride was added to quench the reaction until the solution became colourless. cis-1,2-Dichloroethylene (0.41 mL, 5.50 mmol) was added with a syringe. The reaction was stirred for 4 hours and then the ammonia was evaporated at 25° C. The remaining solid was dissolved in a mixture of water (10 mL) and ethyl acetate (10 mL). The ethyl acetate phase was separated, washed with water (10 mL), dried (MgSO₄) and concentrated to afford {2-[2-(2-tert-butyoxycarbonylamino-ethylsulfanyl)-vinylsulfanyl]-ethyl}carbamic acid tert-butyl ester 8 (2.2 g, yield=96%). The product was pure enough for further reactions. Mp 124° C.; ¹H NMR (300 MHz, DMSO-d₆) δ 6.95 (t, 2H), 6.20 (s, 2H), 3.06-3.12 (m, 4H), 2.74 (t, J=7.5, 4H), 1.36 (s, 9H); ¹³C NMR δ 155.4, 123.0, 77.8, 32.6, 28.2; MS (APCI) m/z (%) 379 (M+H⁺, 50).

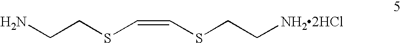

To a solution of the Boc protected linker 8 (0.50 g, 1.30 mmol) in dioxane (8 mL) was added a solution of HCl in dioxane (10 mL) and the solution was stirred at 25° C. for 1 h. After filtration, the crude product was dissolved in methanol, and precipitated with CH₂Cl₂. The product was filtered and dried to give 2-[2-(2-amino-ethylsulfanyl)-vinylsulfanyl]-ethylamine 5 as the di-hydrochloric acid salt (0.28 g, 86%) as a white solid. Mp 155° C.; ¹H NMR (300 MHz, MeOH-d₄) δ 6.31 (s, 2H), 3.15 (t, J=7.0, 4H), 3.01 (t, J=7.5, 4H); ¹³C NMR δ 124.9, 40.6, 31.7; MS (APCI) m/z (%) 179 (M+H⁺, 65).

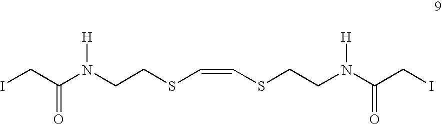

To a solution of diamine 5 (0.20 g, 0.80 mmol) in 0.1 M sodium hydroxide aqueous solution (40 mL) was added a solution of iodoacetic anhydride (0.78 g, 2.20 mmol in 1,2-dichloroethane (10 mL). The mixture was vortexed for 2 minutes. The product was formed as a white precipitate and was filtered out by a frit funnel. The solid was washed with water (10 mL) and dried to produce 2-iodo-N-(2-{2-[2-(2-iodo-acetylamino)-ethylsulfanyl]vinylsulfanyl}-ethyl)-acetamide 9 (0.34 g, 85%) as a white solid. Mp 129° C. ¹H NMR (300 MHz, DMSO-d₆) δ 8.45 (bs, 2H), 6.25 (s, 2H), 3.64 (s, 4H), 3.25 (m, 4H), 2.88 (t, J=6.3, 4H)

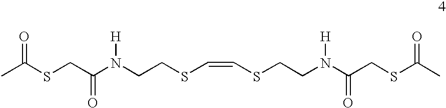

A solution of diamide 9 (0.22 g, 0.43 mmol) and potassium thioacetate (0.11 g, 0.95 mmol) in methanol (80 mL) was evacuated and purged with argon for three times, and then stirred at 50° C. for 4 hours. The methanol was removed under vacuum and the residue was extracted with ethyl acetate (20 mL) and water (20 mL). The organic phase was washed with water (10 mL), dried (MgSO₄), and concentrated to give thioacetic acid S-[(2-{2-[2-(2-acetylsulfanyl-acetylamino)-ethylsulfanyl]-vinylsulfanyl}-ethylcarbamoyl)-methyl] ester 4 (0.15 gm, 84%) as a white solid. Mp 129° C.; ¹H NMR (300 MHz, MeOH-d₄) δ 6.18 (s, 2H), 3.61 (s, 4H), 3.37 (t, J=6.8, 4H), 2.80 (t, J=7.0, 4H), 2.46 (s, 6H); ¹³C NMR δ 195.5, 168.3, 124.7, 39.6, 33.6, 33.0, 30.3; MS (APCI) m/z (%) 411 (M+H⁺, 100)

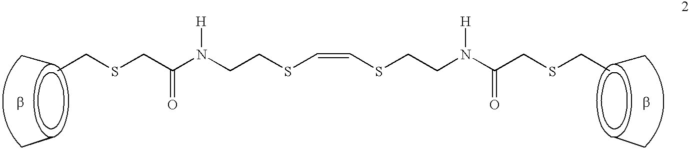

Dithioacetate 4 (0.20 g, 0.58 mmol) and potassium hydroxide (0.10 g, 1.79 mmol) were dissolved in methanol (100 mL). The solution was evacuated and purged with argon three times. The solution was stirred at 50° C. for 10 minutes and then the methanol was removed under vacuum. The residue was dissolved in DMF (40 mL) and 6-monoiodo-β-cyclodextrin (1.00 g, 8.04 mmol) was added. The reaction was stirred at 50° C. for 18 h, then poured into acetone (1 L). The resultant precipitate was filtered and purified by reverse-phase column using a H₂O/MeOH solvent gradient (MeOH 20-80%). This gave pure dimer 2 (0.23 g, 22%) as a white solid. ¹H NMR (300 MHz, DMSO-d₆) δ 8.09 (t, 2H), 6.22 (s, 2H), 5.90-5.60 (m, 28H), 4.95-4.75 (m, 14H), 4.55-4.40 (m, 12H), 3.90-3.45 (m, 56H), 2.77 (t, 4H); MS (FAB): 2560 (M+2⁺, 10).

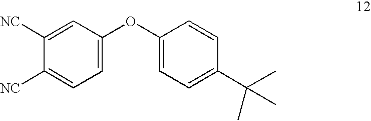

To a solution of 4-tert-butylphenol 10 (2.26 g, 15.0 mmol) in dry DMSO (15 mL) was added 4-nitrophthalonitrile 11 (1.30 g, 7.50 mmol) and potassium carbonate (2.07 g, 15.0 mmol). The reaction was stirred for 20 h at 25° C. The crude product was precipitated by pouring the solution into 150 mL of cold dilute HCl (5 mL of 37% HCl in 150 mL of water), filtered and washed with water until the washings were neutral pH. The crude product was dissolved in CH₂Cl₂ (100 mL) and washed with 5% NaOH solution (5×75 mL) and water (75 mL). The resulting solution was dried (MgSO₄), filtered and concentrated to give 4-(4-teret-butyl-phenoxy)-phthalonitrile 12 (1.52 g, 73%) as a white solid. TLC R_{f} 0.22 1:4 Ethyl acetate:hexanes; Mp 122° C. (lit.^{x} 120° C.); ¹H NMR (300 MHz, CDCl₃) δ 7.72 (dd, J=7.3, 1.9, 2H), 7.47 (dd, J=6.7, 2.2, 2H), 7.27 (s, 1H), 7.24 (d, J=2.6, 1H), 7.00 (dd, J=6.7, 1.1, 2H), 1.36 (s, 9H); ¹³C NMR (75 MHz, CDCl₃) δ 162.1, 151.1, 149.4, 135.4, 127.5, 121.4, 121.3, 120.1, 117.5, 115.5, 115.1, 108.5, 34.6, 31.4; MS (APCI) m/z (%) 277 (M+H⁺, 5).

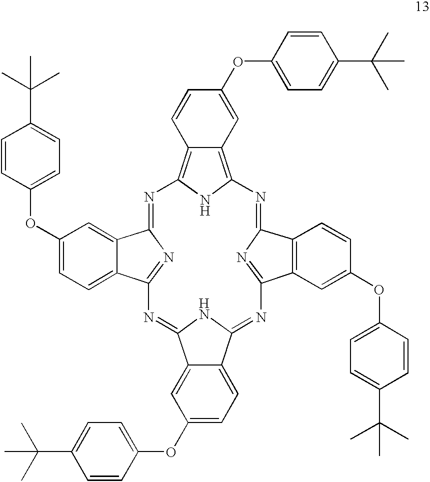

To a solution of lithium pentyloxide (prepared from reaction of lithium (25 mg) with pentanol (4.0 mL), at 140° C. was added dinitrile 12 (0.25 g, 0.91 mmol). The reaction mixture was heated to 140° C. for 3 h. The mixture was allowed to cool and the solution was concentrated The resulting dark mass was dissolved in DMF containing a small amount of methanolic KOH (10 mL) and poured into acetone (10 mL). The precipitate was filtered, treated with concentrated HCl (10 mL), dissolved in acetone (75 mL) and treated with dilute ammonia (75 mL). The resulting precipitate was filtered and dried to afford pure phthalocyanine 13 (57 mg, 23%) as a blue/green solid. ¹H NMR (300 MHz, CDCl₃) δ 8.81-8.67 (m, 4H), 8.63-8.39 (m, 4H), 7.63-7.54 (m, 10H), 7.45-7.33 (m, 7H), 7.29-7.24 (m, 3H), 1.48-1.43 (m, 36H), 3.00 (bs, 2H); MS (FAB) m/z (%) 1107 (M+H⁺, 0.0.5)

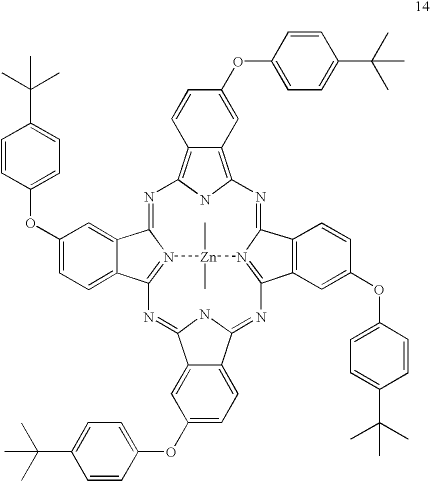

A mixture of phthalocyanine 13 (57 mg, 5.15×10⁵ mol) and zinc acetate dihydrate (25 mg, 1.14×10⁻⁴ mol) in DMF (7 mL) was heated at 70° C. for 20 h. The DMF was removed under vacuum and the resulting solid washed with water (10 mL). TLC analysis (0.2% MeOH (10% NH₃):CHCl₃) showed there to be no other compounds in the resulting solid. The product 14 was isolated as a blue/green solid (60 mg, 100%). TLC R_{f} 0.67 10% MeOH (10% NH₃):CHCl₃; ¹H NMR (300 MHz, CDCl₃) δ 8.72-7.78 (m, 8H), 7.60-7.51 (m, 4H), 7.38-7.07 (m, 16H), 1.55-1.42 (m, 36H; MS (FAB) m/z (%) 1170 (M+H⁺, 2)); λₘₐₓ (nm) 680.

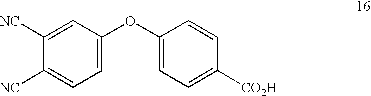

To a suspension of K₂CO₃ (3.50 g, 25 mmol) in dry DMSO (30 mL) was added 4-nitrophthalonitrile 11 (2.00 g, 11.5 mmol) and p-hydroxybenzoic acid (2.36 g, 17.1 mmol). Further K₂CO₃ (3.50 g, 25 mmol) were added after 3 h and after 24 h. The suspension was stirred at 25° C. for 5 days. The suspension was added to water (600 mL) and the pH was adjusted to 1 using concentrated HCl. The resulting precipitate was recrystallised from methanol (50 mL) to give pure 4-(3,4-dicyano-phenoxy)-benzoic acid 16 (2.65 g, 87%) as a white solid. ¹H NMR (300 MHz, CDCl₃) δ 8.16 (d, J=8.7, 1H), 8.03 (d, J=8.8, 2H), 7.94 (d, J=2.5, 1H), 7.54 (dd, J=8.7, 2.5, 1H), 7.27 (d, J=8.8, 2H); MS (APCI) m/z (%) 297 (M+MeOH⁺, 60).

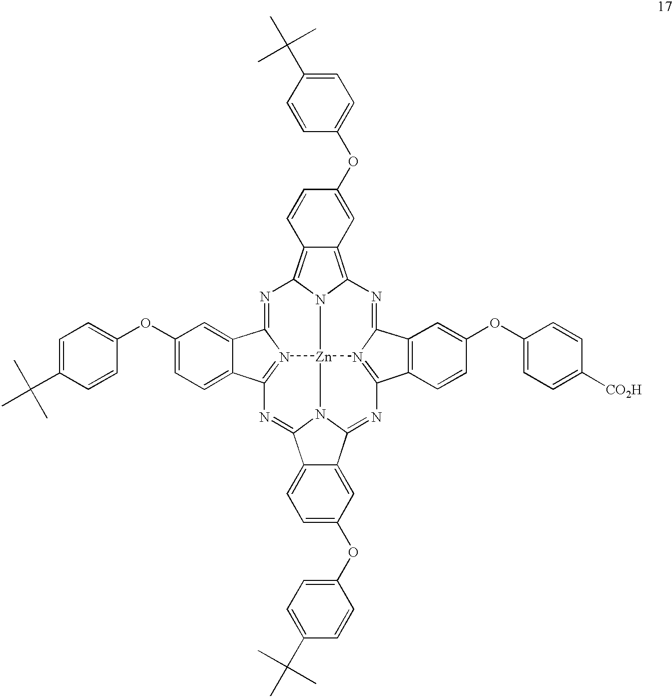

To a suspension of benzoic acid dinitrile 16 (0.19 g, 0.73 mmol) and tert-butyl dinitrile (0.30 g, 1.08 mmol) in pentanol (10 mL) at 140° C. was added lithium (0.10 g, 14.5 mmol) and the mixture was stirred at 140° C. for 15 min. Once cooled to 25° C., glacial acetic acid (30 mL) was added, the resultant precipitate was centrifuged and washed with water (10 mL). The products were dissolved in DMF (30 mL), zinc acetate dihydrate (0.12 g, 0.55 mmol) was added and the reaction was heated at 80° C. for 15 h. The solution was cooled to 25° C., the DMF was removed under vacuum and the resulting solid was washed with water (20 mL). This solid was dissolved in the minimum volume of DMF and was purified by column chromatography on silica, starting with an eluent of diethyl ether:DMF 98:2. Once the first fraction, the tetra-tert-butyl phthalocyanine 14, had come off, the eluent was very slowly changed to pure DMF. The next fraction to come off was the desired complex 17, which was isolated as a blue/green solid (88 mg, 21%). ¹H NMR (300 MHz, CDCl₃) 9.22-8.60 (m, 8H) 8.25-7.35 (m, 20H), 1.46-1.35 (m, 27H); MS (FAB) m/z (%) 1157 (M+H⁺, 5).

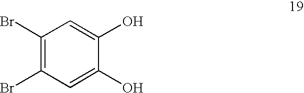

To a solution of catechol 18 (11.00 g, 0.10 mol) in glacial acetic acid (50 mL) at 0° C. was very slowly added a solution of bromine (11 mL, 34 g, 0.21 mol) in glacial acetic acid (50 mL). This mixture was stirred at 25° C. for 18 h and after this TLC (60:40 Hexane:Ethyl Acetate) suggested that there was little starting catechol 18 left. The HBr and glacial acetic acid were removed by distillation under water-pump vacuum. The dark residue was quenched with ice water (350 mL). The resulting precipitate was filtered, dried in vacuum and recrystallised from benzene to give three crops of crystals, giving 4,5-dibromocatechol 19 (15.37 g, 57%) as a white solid. TLC R_{f} 0.34 2:3 Ethyl acetate:hexanes; Mp 110-111° C. (lit.^{x} 119-121° C.); ¹H NMR (300 MHz, CDCl₃) δ 7.14 (s, 2H), 5.30 (s, 2H) ¹³C NMR (75 MHz, CDCl₃) δ; MS (APCI) m/z (%) 265 (M−H⁺, 25).

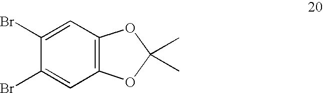

To a mixture of 4,5-dibromocatechol 19 (10.72 g, 0.04 mol) and P₂O₅ (1.14 g, 8.00 mmol) in toluene (20 mL) heated at 75° C. was added dropwise acetone (5.90 mL, 0.08 mol). During the addition, every 30 min P₂O₅ (1.14 g, 8.00 mmol) was added, for 2 h (4 portions). The mixture was then stirred for 1 h. Once cooled to 25° C., the viscous oil was poured into 25% NaOH in water (25 mL), after which a white precipitate was seen to form. This precipitate was dissolved by addition of water (50 mL) to the solution. The aqueous layer was washed with water (3×50 mL) dried (MgSO₄) and concentrated to yield pure 5,6-dibromo-2,2-dimethyl-benzo[1,3]dioxole 20 (1.13 g, 9%) as a white solid. TLC R_{f} 0.631:4 Ethyl acetate:hexanes; Mp 93-94° C.; ¹H NMR (300 MHz, CDCl₃) δ 6.97 (s, 2H), 1.66 (s, 6H); ¹³C NMR (75 MHz, CDCl₃) δ 147.9, 120.2, 114.7, 113.1, 25.9.

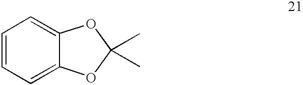

To a mixture of catechol 18 (11.00 g, 0.10 mol) and P₂O₅ (2.84 g, 20 mmol) in toluene (50 mL) at 75° C. was added dropwise acetone (14.7 mL, 0.2 mol) over 2 h. After the addition had been started, P₂O₅ (2.84 g, 20 mmol) was added to the reaction mixture every 30 min, total 3 portions. After 2 h, further acetone (7.3 mL, 0.1 mol) was added dropwise. The reaction was then stirred for a further 1 h and was then cooled to 25° C. The organic solution was carefully decanted and to this was added 25% NaOH in water (15 mL). The organic layer was separated, washed with water (2×20 mL) and concentrated. The resulting oil was distilled under high vacuum to give 1,2-isopropylidenedioxybenzene (2,2-dimethyl-benzo[1,3]dioxole) 21 (5.00 g, 33%) as a clear liquid. ¹H NMR (300 MHz, CDCl₃) δ 6.80-6.72 (m, 4H), 1.67 (s, 6H); ¹³C NMR (75 MHz, CDCl₃) δ 147.4, 121.1, 117.4, 108.5, 25.9; MS (APCI) m/z (%) 265 (M−H⁺, 25).

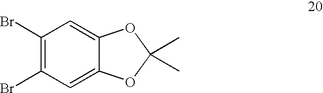

To a solution of 1,2-isopropylidenedioxybenzene 21 (4.80 g, 31.9 mmol) in DMF (60 mL) was added portionwise N-bromosuccinimide (11.94 g, 67.1 mmol) at 25° C. The light yellow solution was stirred at 25° C. for 24 h. The solution was then poured into water (600 mL) and extracted with CH₂Cl₂ (7×150 mL). The combined organic extracts were washed with water (4×300 mL), dried (MgSO₄), filtered and concentrated. The resulting solid was recrystallised from warm benzene to give pure 5,6-dibromo-2,2-dimethyl-benzo[1,3]dioxole 20 (4.94 g, 50%) as a white solid. TLC R_{f} 0.63 1:4 Ethyl acetate:hexanes; Mp 93-94° C.; ¹H NMR (300 MHz, CDCl₃) δ 6.97 (s, 2H), 1.66 (s, 6H); C NMR (75 MHz, CDCl₃) δ 147.9, 120.2, 114.7, 113.1, 25.9.

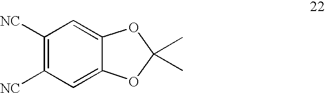

A mixture of 20 (4.94 g, 16.0 mmol), CuCN (5.77 g, 654.2 mmol) in dry DMF (65 mL) was heated to 155° C. for 5 h. The dark mixture was treated with ammonia water (250 mL) and stirred for 30 min. The mixture was then filtered, washed with water (100 mL) and dried in air for 18 h. The resulting solid was extracted with diethyl ether using a Soxlet extractor for 3 days. The solvent was then removed and the residue twice recrystallised from benzene to give 2,2-dimethyl-benzo[1,3]dioxole-5,6-dicarbonitrile 22 (1.58 g, 49%) as a white solid. Mp 202-203° C.; ¹H NMR (300 MHz, CDCl₃) δ 7.06 (s, 2H), 1.77 (s, 6H; MS (APCI) m/z (%) 265 (M+MeOH⁺, 70), 233 (M+MeOH⁺, 100), 218 (M+H₂O⁺, 20), 201 (M+H⁺, 4).

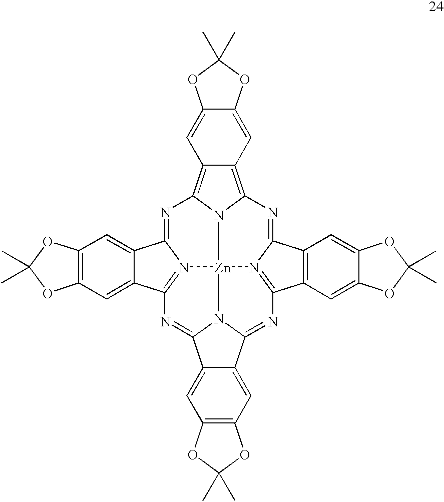

To a solution of 3,4-dicyano-1,2-isopropylidenedioxybenzene 22 (0.20 g, 1.00 mmol) in pentanol (4 mL) at 140° C. was added lithium (0.10 g, 14.5 mmol). The reaction was heated at 140° C. for 2 h, cooled and glacial acetic acid (15 mL) added. The solvents were then removed under vacuum. The light green solid was dissolved in DMF (30 mL), treated with zinc acetate dihydrate (0.11 g, 0.50 mmol) and heated at 65° C. for 15 h. The green coloured solution was concentrated and washed with water (10 mL). TLC (10% MeOH (10% NH₃):CHCl₃) showed there still to be some starting unmetallated phthalocyanine left. This solid was dissolved in DMF (30 mL) and was treated with zinc acetate dihydrate (0.11 g, 0.50 mmol) and potassium carbonate (0.08 g, 0.55 mmol) (to aid complexation). This mixture was heated at 65° C. for 20 h. The green coloured solution was cooled to 25° C. and concentrated under vacuum. The resulting solid was dissolved in CHCl₃ (30 mL) and carefully added to a silica column packed in CHCl₃. The eluent was gradually changed to 10% MeOH (10% NH₃):CHCl₃ which gave more pure, but not completely pure material. The fractions containing the desired material were concentrated and dissolved in CHCl₃ (20 mL). This solution was carefully added to a silica column packed in CHCl₃ The eluent was gradually changed to 1.5% MeOH (10% NH₃):CHCl₃ and then very slowly changed to 5% MeOH (10% NH₃):CHCl₃. Early fractions contained the desired product but were strongly contaminated with impurities. Subsequent fractions contained the pure compound, affording pure phthalocyanine 24 (0.13 g, 60%) as a green solid. ¹H NMR (300 MHz, CDCl₃) δ 7.15-7.05 (m, 8H), 1.81-1.73 (m, 24H); MS (APCI) m/z (%) 864 (M⁺, 4); λₘₐₓ (nm) 667.

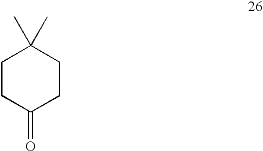

A mixture of 4,4-dimethyl-2-cyclohexenone 25 (5.00 g, 40.26 mmol) and 10% Pd/C (0.10 g) in pentane (70 mL) was hydrogenated at 5° C. at 1 atmosphere of H₂. The reaction was allowed to warm to 25° C. for 18 h. The mixture was filtered through Celite, concentrated to 15 mL and cooled in an i-PrOH-dry ice bath. Filtration afforded 4,4-dimethylcyclohexanone 26 (2.60 g, 51%) as a white fluffy solid. ¹H NMR (300 MHz, CDCl₃) δ 2.35 t, J=7.0, 4H), 1.67 (t, J=7.0, 4H), 1.10 (s, 6H); C NMR (75 MHz, CDCl₃) δ 212.6, 39.2, 38.0, 29.9, 27.5; MS (APCI) m/z (%) 159 (M+MeOH⁺, 20), 127 (M+H⁺, 100).

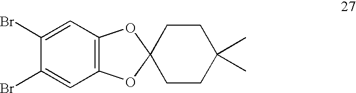

A solution of 3,4-dibromocatechol 19 (2.00 g, 7.46 mmol) and 4,4-dimethylcyclohexanone 26 (0.94 g, 7.46 mmol) in toluene (22 mL) containing p-toluene sulfonic acid mono-hydrate (0.074 g, 0.39 mmol) was set up with a Dean-Stark head, and was heated at 130° C. for 15 h. Once cooled to 25° C., the solution was washed with aqueous NaHCO₃ solution (10 mL), water (10 mL), dried (MgSO₄), filtered and concentrated. The mixture was shown by ¹H NMR to still contain catechol, and so the mixture was dissolved in diethyl ether (25 mL). This solution was washed with 10% NaOH solution (3×20 mL), water (20 mL), dried (MgSO₄), filtered and concentrated. The resulting yellow solid was recrystallised from hexanes to give 4,5-dibromo-1,2-di-O-(4′,4′-dimethylcyclohexylidene)catechol 27 (0.71 g, 25%). Mp 126-127° C.; ¹H NMR (300 MHz, CDCl₃) δ 6.97 (s, 2H), 1.91 t, J=6.0, 4H), 1.51 (t, J=6.0, 4H), 0.99 (s, 6H); ¹³C NMR (75 MHz, CDCl₃) δ 147.9, 121.3, 114.5, 113.1, 35.7, 31.4, 29.3, 27.8; MS (EI) m/z (%) 376 (M⁺, 100), 314 (50), 304 (50).

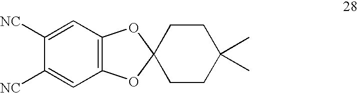

A mixture of 27 (0.64 g, 1.70 mmol), CuCN (0.61 g, 6.81 mmol) in DMF (7 mL) was heated to 150° C. for 5 h. The mixture was then cooled to 25° C., treated with ammonia water (25 mL) and stirred for 30 min. The mixture was filtered, washed with water (10 mL) and air dried for 18 h. The resulting solid was extracted with diethyl ether using a Soxlet extractor for 3 days. The solvent was then evaporated to produce 4,5-dicyano-1,2-di-O-(4′,4′-dimethylcyclohexylidene)catechol 28 (1.58 g, 49%) as a white solid. Mp 159° C.; ¹H NMR (300 MHz, CDCl₃) δ 7.05 (s, 2H), 1.97 (s, 4H), 1.56 (s, 4H), 1.02 (s, 6H); ¹³C NMR (75 MHz, ClCl₃) δ 151.4, 124.4, 115.8, 112.5, 110.0, 35.5, 31.5, 29.3, 27.7; MS (EI) m/z (%) 268 (M⁺, 100), 252 (30), 212 (70), 197 (100).

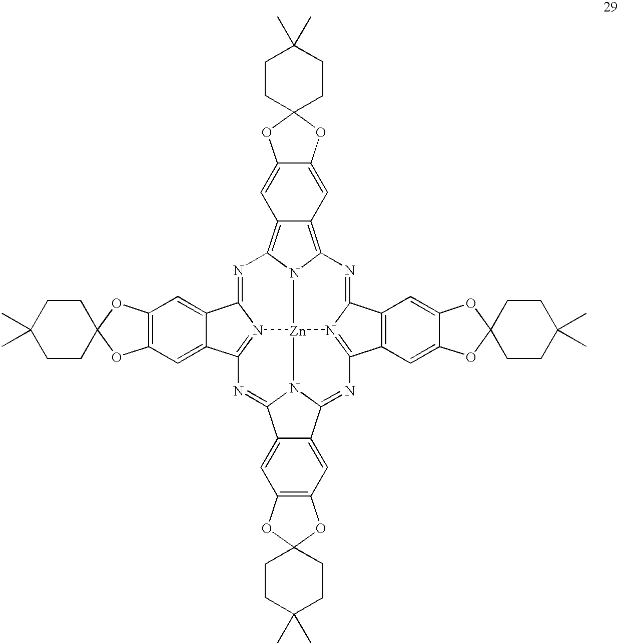

To a solution of 28 (90 mg, 0.33 mmol) and anhydrous 140° C. was added lithium (33 mg, 4.75 mmol). This mixture was heated at 140° C. for 17 h. The reaction was cooled to 25° C., solvent was removed under vacuum and washed with water (10 mL). The resulting solid was dissolved in CHCl₃ and purified on a silica column (CHCl₃—1% MeOH (10% NH₃):CHCl₃) to give the desired phthalocyanine 29 (55 mg, 60%) as a blue/green solid. ¹H NMR (300 MHz, CDCl₃) δ 8.15 (bs, 8H), 2.35 (bs, 16H), 1.88 (bs, 16H), 1.24 (*s, 26H); MS (FAB) m/z (%) 1137 (M+H⁺, 6), 1136 (M⁺, 5) λₘₐₓ (nm) 669.

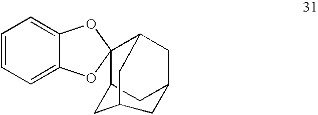

A mixture of catechol 18 (4.03 g, 36.6 mmol), 2-adamantanone 30 (5.00 g, 33.3 mmol) and p-toluene sulfonic acid mono-hydrate (0.13 g) in benzene (125 mL) was heated at 80° C. for 22 h. The solution was cooled to 25° C. and washed successively with 10% NaOH solution (3×50 mL), water (50 mL), dried (MgSO₄), filtered and concentrated. The resulting light yellow solid was recrystallised from benzene to give 1,2-di-O-(adamantylidene)catechol 31 (6.77 g, 84%) as an off-white solid. Mp 130° C. (lit.^{x} 126-127° C.); ¹H NMR (300 MHz, CDCl₃) δ 6.75 (s, 4H), 2.18-1.75 (m, 14H); ¹³C NMR (75 MHz, CDCl₃) δ 147.7, 120.8, 108.4, 37.1, 36.7, 34.4, 26.7; MS (FAB) m/z (%) 242 (M⁺, 95).

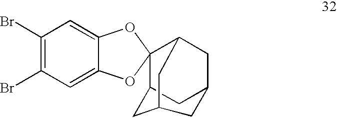

A solution of 31 (2.42 g, 10 mmol) in CCl₄ (10 mL) cooled to 0° C. was treated with Br₂ (3.20 g, 1.02 mL, 20 mmol) dissolved in CCl₄ (1 mL). The mixture was stirred at 0° C. for 30 min. The mixture was diluted with CHCl₃ (29 mL) and washed with 10% aqueous NaOH solution (2×20 mL), water (20 mL), dried (MgSO₄), filtered and concentrated. The resulting solid was recrystallised twice from hexanes to give pure 4,5-dibromo-1,2-di-O-(adamantylidene)catechol 32 (1.92 g, 48%) as a white solid. TLC R_{f} 0.75 1:19 Ethyl acetate:hexanes; Mp 195° C.; ¹H NMR (300 MHz, CDCl₃) δ 6.99 (s, 2H), 2.16-1.75 (m, 14H); ¹³C NMR (75 MHz, CDCl₃) δ 148.2, 124.0, 114.3, 113.0, 36.9, 36.8, 34.3, 26.5; MS (FAB) m/z (%) 400 (M⁺, 25).

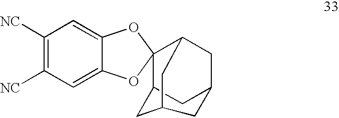

A mixture of 32 (1.43 g, 3.57 mmol) and CuCN (1.28 g, 14.30 mmol) in DMF (15 mL) was heated to 150° C. for 3 h. The mixture was then cooled to 25° C., treated with ammonia water (50 mL) and stirred for 30 min. The mixture was filtered, washed with water (10 mL) and air dried for 18 h. The resulting solid was extracted with dithyl ether using a Soxlet extractor for 3 days. The solvent was then evaporated and the resulting solid was recrystallised from warm benzene to give 4,5-dicyano-1,2-di-O-(adamantylidene)catechol 33 (0.48 g, 46%) as a green solid. Mp 210° C.; ¹H NMR (300 MHz, CDCl₃) δ 7.06 (s, 2H), 2.21-1.79 (m, 14H); ¹³C NMR (75 MHz, CDCl₃) δ 151.6, 127.2, 115.8, 112.4, 109.9, 37.0, 36.7, 34.2, 26.3; MS (FAB) m/z (%) 315 (M+Na+, 10), 293 (M+H+, 20).

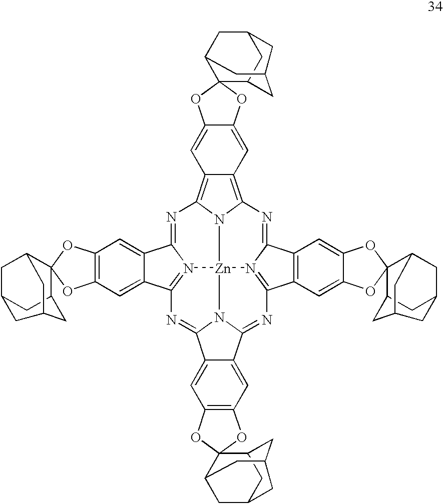

To a solution of phthalonitrile 33 (0.24 g, 0.82 mmol) and anhydrous zinc acetate (75 mg, 0.41 mmol) in pentanol (5 mL) at 140° C. was added lithium (80 mg, 11.5 mmol). The solution was heated at 140° C. for 18 h. The solution was then cooled to 25° C. and TLC analysis (10% MeOH (10% NH₃):CHCl₃) showed there to be only the desired phthalocyanine 34 present. TLC Rf 0.80 10% MeOH (10% NH₃):CHCl₃; 1H NMR (300 MHz, CDCl₃) δ 8.60 (bs, 8H), 1.61-2.67 (m, 56H); MS (FAB) m/z (%) 1235 (M+H+, 5); λₘₐₓ (nm) 668.

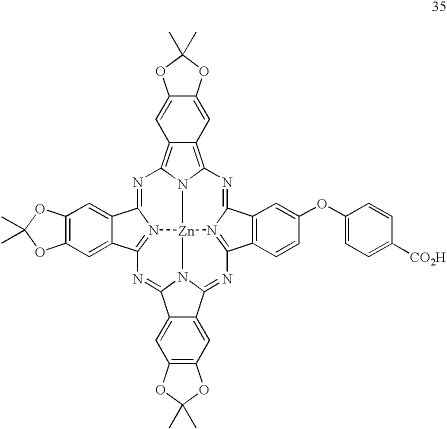

To a solution of 3,4-dicyano-1,2-isopropylidenedioxybenzene 22 (0.21 g, 1.08 mmol), 16 (0.19 g, 0.73 mmol) and anhydrous zinc acetate (0.10 g, 0.55 mmol) in pentanol (10 mL) at 140° C. was added lithium (100 mg, 14 mmol). The reaction was heated at 140° C. for 20 h, cooled to 25° C. and concentrated under vacuum. The resulting solid was dissolved in the minimum volume of DMF and added to a silica column packed using diethyl ether:DMF 98:2. The eluent diethyl ether:DMF 98:2 was used until all of the first compound to come off the column had come off. The eluent was then very gradually changed to DMF. At this time the next fraction came off, which contained the desired compound. 3:1 Mixed phthalonitrile 35 (72 mg, 22%) was isolated as a blue/green solid. TLC Rf 0.24 20% MeOH (10% NH₃):CHCl₃; ¹H NMR (300 MHz, DMSO-d₆) δ 8.71 (m, 4H), 8.13-8.01 (m, 5H), 7.71-7.23 (m, 4H) 5.76 (s, 1H), 1.96 (s, 18H); MS (FAB) m/z (%) 930 (M+H+, 2) 929 (M+, 2); λₘₐₓ (nm) 666.

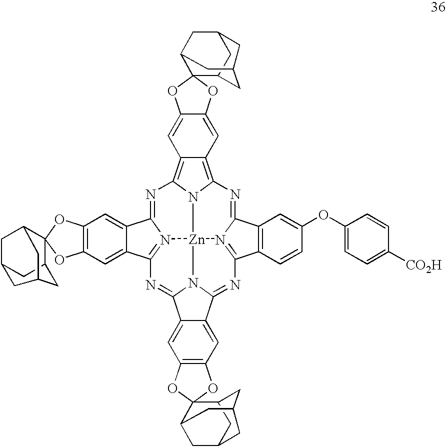

To a solution of phthalonitrile 33 (0.12 g, 0.41 mmol), 16 (72 mg, 0.28 mmol) and anhydrous zinc acetate (38 mg, 0.21 mmol) in pentanol (4 mL) at 140° C. was added lithium (40 mg, 5.30 mmol). The reaction was heated at 140° C. for 17 h, the solution was then cooled to 25° C. and the solvent removed under vacuum. The resulting solid was dissolved in the minimum volume of DMF and added to a silica column packed using diethyl ether:DMF 98:2. The eluent diethyl ether:DMF 98:2 was used until all of the first compound to come off the column had come off. The eluent was then very gradually changed to DMF. At this time the next fraction came off, which contained the desired compound which was slightly contaminated. The relevant fractions were concentrated, the resulting solid was dissolved in the minimum volume of DMF and added to a silica column packed using diethyl ether:DMF 98:2. The eluent diethyl ether:DMF 98:2 was used until all of the first compound to come off the column had come off. The eluent was then very gradually changed to DMF. 3:1 Mixed phthalonitrile 36 (68 mg, 41%) was isolated as a blue/green solid. TLC R_{f} 0.25 20% MeOH (10% NH₃):CHCl₃; ¹H NMR (300 MHz, DMSO-d₆) δ 9.38-9.33 (m, 1H), 8.94-8.90 (m, 1H), 8.58-8.42 (m, 4H), 8.16 (d, J=8.4, 2H), 8.04-7.92 (m, 1H), 7.58 (s, 1H) 7.49 (d, J=8.7, 2H), 7.27 (s, 1H), 2.33-1.73 (m, 42H); MS (FAB) m/z (%) 1205 (M⁺, 0.5); λₘₐₓ (nm) 667.

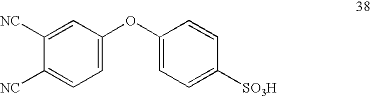

To a solution of 4-nitrophthalonitrile 11 (1.73 g, 10.00 mmol) in dry DMSO (20 mL) was added 4-hydroxy benzensulfonic acid sodium salt dihydrate (3.98 g, 15.0 mmol), potassium carbonate (2.07 g, 15.00 mmol) and 4 Å molecular sieves. Further potassium carbonate (2.07 g, 15.00 mmol) was added after 4 h. The reaction was stirred for 3 days. The mixture was poured into water (150 mL) and the pH of the solution adjusted to 0 using HCl. The solution was then carefully evaporated until a precipitate was seen to form. The resulting solid was filtered and washed with ethanol (50 mL) to give pure 4-(3,4-dicyano-phenoxy)-benzenesulfonic acid 38 (2.10 g, 70%) as a white solid. TLC R_{f} 0.67 7:7:5 i-PrOH:Ethyl Acetate:Water; ¹H NMR (300 MHz, DMSO-d₆) δ 8.11 (d, J=8.7, 1H), 7.86 (J=2.5, 1H), 7.72 (ap. dt, J=8.7, 2.6, 2H), 7.41 (dd, J=8.7, 2.6, 1H), 7.14 (ap. dt, J=8.7, 2.6, 2H); MS (FAB) m/z (°) 299 (M−H⁺, 100).

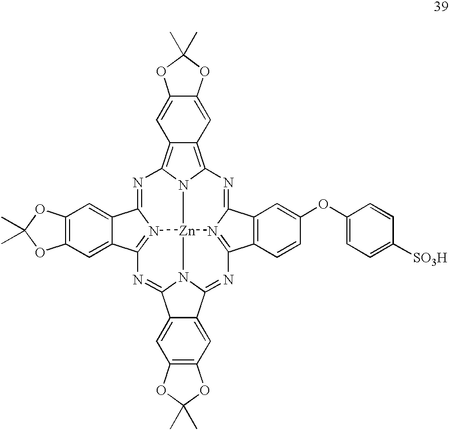

To a solution of 3,4-dicyano-1,2-isopropylidenedioxybenzene 22 (0.105 g, 0.53 mmol), 38 (0.11 g, 0.37 mmol) and anhydrous zinc acetate (50 mg, 0.28 mmol) in pentanol (5 mL) at 140° C. was added lithium (50 mg, 7 mmol). The reaction was heated at 140° C. for 22 h, then cooled to 25° C. and concentrated under vacuum. The resulting solid was dissolved in a mixture of MeOH:DMF:10% ammonium formate buffer (65:25:10), the same solvent mixture was also used to pack a reverse-phase silica column. The reaction mixture was eluted with this system, yielding some fractions containing improved purity product. These were concentrated, dissolved in the same MeOH:DMF:10% ammonium formate buffer (65:25:10), the same solvent mixture was also used to pack a reverse-phase silica column. The product could now be isolated pure 39 (75 mg, 44%). TLC R_{f} 0.20 20% MeOH (10% NH₃):CHCl₃; ¹H NMR (300 MHz, DMSO-d₆) δ 9.42-9.36 (m, 1H), 8.72-8.50 (m, 4H), 8.41-8.21 (m, 1H), 7.78 (d, J=8.7, 2H), 7.64-7.53 (m, 1H), 7.32 (d, J=9.0, 2H), 7.25 (s, 1H), 7.11 (s, 1H), 1.98-1.95 (m, 6H), 1.71-1.65 (m, 12H); MS (FAAB) m/z (%) 966 (M+H⁺, 1.5), 965 (M⁺, 1.5); λₘₐₓ (nm) 665.

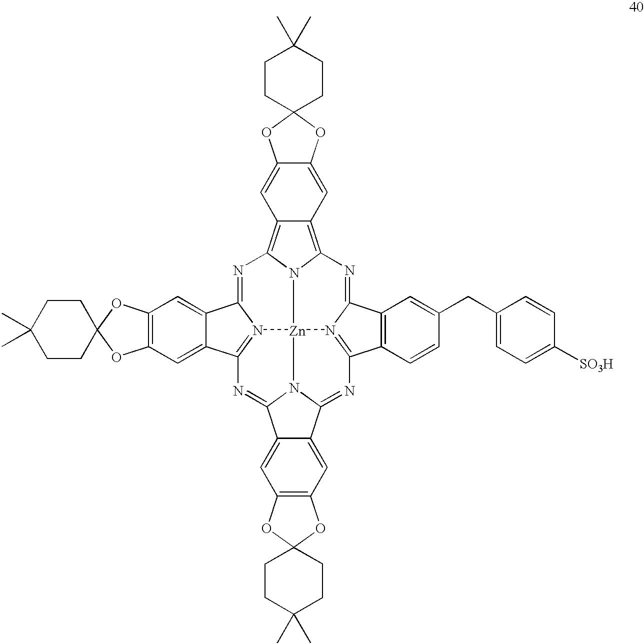

To a solution of 28 (50 mg, 0.186 mmol), 38 (38 mg, 0.125 mmol) and anhydrous zinc acetate (17 mg, 0.093 mmol) in pentanol (2 mL) at 140° C. was added lithium (17 mg, 2.43 mmol). The reaction was heated at 140° C. for 18 h, cooled to 25° C. and concentrated under vacuum. The resulting solid was dissolved in a mixture of MeOH: DMF: 10% ammonium formate buffer (60:20:20), the same solvent mixture was also used to pack a reverse-phase silica column. The column was eluted with this system, which allowed the more polar compounds to come off whilst leaving the desired compound still on the column. Once the more polar compounds had finished coming off the solvent system was changed to MeOH: DMF: THF (55:25:20) and this gave almost pure product. These fractions were concentrated, dissolved in the MeOH:DMF:10% ammonium formate buffer (60:20:20), the same solvent mixture was also used to pack a reverse-phase silica column. The column was eluted with this system, which allowed the more polar compounds to come off whilst leaving the desired compound still on the column. Once the more polar compounds had finished coming off, the solvent system was changed to MeOH: DMF: THF (55:25:20) and this gave pure phthalocyanine 40 (26 mg, 36%) as a blue/green solid. TLC R_{f} 0.21 20% MeOH (10% NH₃):CHCl₃; ¹H NMR (300 MHz, DMSO-d₆) δ 9.38-9.28 (m, 1H), 8.89-8.82 (m, 1H), 8.67-8.49 (m, 4H), 7.82 (d, J=7.5, 2H), 7.37 (d, J=8.1, 2H), 7.25 (d, J=7.2, 1H), 7.03 (s, 1H), 6.87 (d, J=7.5, 1H), 2.27 (bs, 4H), 1.96 (bs, 4H), 1.74 (bs, 4H), 1.49 (bs, 4H), 1.22-0.74 (m, 26H); MS (FAB) m/z (%) 1171 (M⁺, 30); λₘₐₓ (nm) 666.

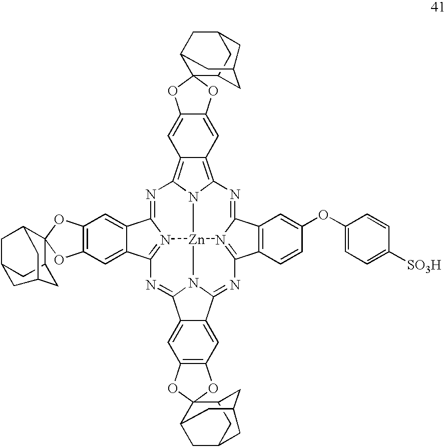

To a solution of 33 (54 mg, 0.186 mmol) 38 (38 mg, 0.125 mmol) and anhydrous zinc acetate (17 mg, 0.093 mmol) in pentanol (2 mL) at 140° C. was added lithium (17 mg, 2.43 mmol). The reaction was heated at 140° C. for 4 h, then cooled to 25° C. and concentrated under vacuum. The resulting solid was dissolved in a mixture of MeOH: DMF: 10% ammonium formate buffer (60:20:20), the same solvent mixture was also used to pack a reverse-phase silica column. The column was eluted with this solvent system, which allowed the more polar compounds to come off whilst leaving the desired compound still on the column. Once the more polar compounds had finished coming off, the solvent system was changed to MeOH:DMF:THF (55:25:20), and this gave pure phthalocyanine 41 (30 mg, 39%) as a blue/green solid. TLC R_{f} 0.25 20% MeOH (10% NH₃):CHCl₃; ¹H NMR (300 MHz, DMSO-d₆) δ 9.378 (bs, 2H) 8.93-8.85 (m, 1H) 8.70-8.52 (m, 3H) 7.80 (d, J=7.5, 2H), 7.54 (s, 1H), 7.50 (d, J=9.9, 1H), 7.37 (d, J=7.5, 2H), 7.05 (s, 1H), 2.26-1.72 (m, 42H); MS (FAB) m/z (%) 1243 (M⁺, 10); λₘₐₓ (nm) 667.

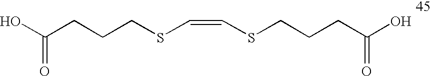

4,4′-Dithioldibutyric acid (2.00 g, 8.38 mmol) was placed in a three-neck flask and was evacuated and purged with argon three times. Liquid ammonia (100 mL) was condensed into the flask using a cold trap. Sodium metal pieces were added until the blue colour remained. This solution was then stirred for 40 min and sodium pieces were added if the colour faded out. The reaction was quenched with the addition of the minimum amount of ammonium chloride, then cis-1,2-dichloroethylene (0.82 g, 8.38 mmol) was added and the reaction was stirred for 4 h. The ammonia was allowed to evaporate off, then the residue was dissolved in water (50 mL) and the solution neutralised using dilute HCl. The mixture was then filtered and dried under vacuum which gave pure 4-[2-(3-carboxy-propylsulfonyl)-vinylsulfanyl]-butyric acid 45 (1.99 g, 90%) as a white solid. Mp 120° C.; ¹H NMR (300 MHz, CDCl₃) δ 12.11 (2, 2H), 6.19 (s, 2H), 2.71 (t, J=7.2, 4H) 2.30 (t, J=7.2, 4H), 1.75 (t, J=7.2, 4H); ¹³C NMR (75 MHz, CDCl₃) δ 174.0, 123.0, 32.3, 32.1, 25.5.

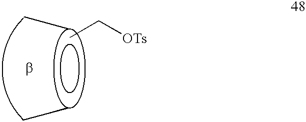

β-Cyclodextrin 47 (40.0 g, 35.3 mmol) was dissolved in hot water (900 mL) (80° C.), then cooled down to 25° C. while being vigorously stirred. p-Toluenesulfonyl imidazole (15.60 g, 70.2 mmol) was added as finely grounded powder. The suspension was stirred for 2 h. Sodium hydroxide (18 g) was dissolved in water (50 mL) and the solution was added to the reaction mixture over a period of 20 minutes. After stirring for another 10 minutes, the mixture was filtered through a frit funnel. The reaction was quenched with ammonium chloride (48.2 g), and the solution was concentrated to about half of its volume. After cooling at 0° C. for an hour, the precipitate was filtered and washed with water (50 mL), acetone (50 mL), then lyophilized to afford the desired product 48 (25 g, 52%) as a white solid. ¹H NMR (300 MHz, DMSO-d₆) δ 7.24(d, 2H), 7.43(d, 2H), 5.60-5.83(m, 14H), 4.74-4.90(m, 7H), 4.30-4.53(m, 6H), 4.29(dd, 1H), 3.40-3.73(m, 28H), 2.41(s, 3H).

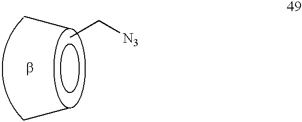

6-Monotosyl-β-cyclodextrin 48 (2.00 g, 1.57 mmol) and sodium azide (2.04 g, 31.4 mmol) were dissolved in DMF (20 mL) and heated to 80° C. for 10 h. The reaction was then cooled to 25° C. and poured into acetone (1 L). The resultant precipitate was filtered and purified by reverse-phase column using a H₂O/MeOH solvent gradient (MeOH:H₂O 0-80%). This gave pure 6-mono-azido-β-cyclodextrin 49 (1.60 g, 91%) as a white solid. ¹H NMR (300 MHz, DMSO-d₆) 5.80-5.62 (m, 13H), 5.60 (d, 1H), 4.87 (d, 1H), 4.85-4.78 (m, 6H), 4.55-4.40 (m, 6H), 3.80-3.46 (m, 28H) 3.46-3.20 (m, overlap with water peak).

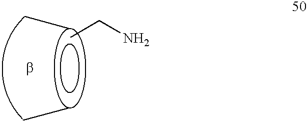

To a solution of 6-mono-azido-β-cyclodextrin 49 (1.00 g, 0.86 mmol) in DMF (20 mL) was added triphenylphosphine (0.50 g, 1.9 mmol) and the reaction was stirred at 90° C. for 18 h. The reaction mixture was then cooled to 25° C. and poured into acetone (1 L). The precipitate was then filtered, washed with acetone (100 mL) and dissolved in water (10 mL). After lyophilisation, the desired 6-monoamino-β-cyclodextrin 50 (0.85 g, 86%) was isolated as a white solid. ¹H NMR (300 MHz, DMSO-d₆) δ 5.90-5.55 (m, 14H), 4.89 (d, 1H), 4.88-4.75 (m, 6H), 4.60-4.35 (m, 6H), 3.75-3.49 (m, 26H), 3.03 (m, 1H), 2.80 (m, 1H); MS (FAB) m/z (%) 1134 (M+H⁺, 10).

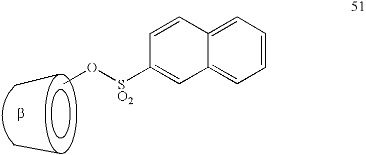

β-Cyclodextrin 47 (20.0 g, 7.6 mmol) was dissolved in a mixture of water (150 mL) and MeCN (50 mL) and its pH was adjusted to 12.0 using 4.0 M NaOH aqueous solution. The solution was heated to 40° C. and was stirred vigorously. 2-Naphthalenesulphonyl chloride (10 g, 44.1 mmol) was added, and the mixture was stirred for another two minutes until its pH dropped to 7. The mixture was then filtered, and the filtrate was diluted with water (1 L) and loaded onto a reverse phase silica column. After elution with water-methanol liner gradient (0-80% MeOH/H₂O), 3-mononapthalenesulfonyl-β-cyclodextrin 51 (3.20 g, 16%) was obtained as a white solid. ¹H NMR (400 MHz, DMSO-d₆) δ 8.68(s, 1H), 8.21 (d, 1H), 8.14 (d, 1H), 8.06 (d, 1H), 7.98 (d, 2H), 7.79-7.67 (m, 2H), 6.02 (d, 1H), 5.88-5.60 (m, 10H), 5.51 (d, 1H), 4.97-4.75 (m, 7H), 4.68 (t, 1H), 4.60-4.48 (m, 5H), 4.34 (t, 1H), 4.16 (d, 1H), 3.88-3.42 (m, 28H), 3.42-3.15 (m, overlap with water peak).

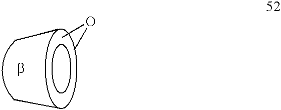

2-Mononaphthalenesulfonyl-β-cyclodextrin 51 (3.20 g, 2.43 mmol) was dissolved in 10% Na₂CO₃ aqueous solution (50 mL) and was stirred at 50° C. for 10 hours. The mixture was loaded on a reverse phase silica column and was eluted with water-methanol gradient (0-80% MeOH/H₂O). The fractions containing the desired product was collected and methanol was removed under reduced pressure to give β-cyclodextrin monoalloepoxide 52 (3.00 g, 96%) as a white solid. ¹H NMR (400 MHz, DMSO-d₆) δ 5.90-5.45 (m, 9H, 5.33 (bs, 1H), 5.23 (d, 1H), 5.19 (d, 1H), 5.07 (bs, 1H), 4.88-4.73 (m, 6H), 4.60 (t, 1H), 4.56-4.32 (m, 6H), 3.90 (d, 1H), 3.82-3.41 (m, 28H), 3.41-3.13 (m, overlap with water peak).

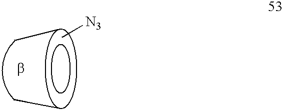

β-Cyclodextrin monoalloepoxide 52 (2.00 g, 1.79 mmol) and sodium azide (0.50 g, 7.69 mmol) were dissolved 0 dry DMF (20 mL) and heated to 90° C. for 18 h. The DMF was removed under vacuum and the residue was dissolved in water, loaded on a reverse phase silica column, and eluted slowly and very carefully with water-methanol linear gradient (methanol 20-80%, v/v). Two fractions (about 1:3) that contained cyclodextrin were collected and the methanol was removed under reduced pressure. The major fraction contained the desired 2-monoazido-β-cyclodextrin 53 (1.20 g, 60%) as a white solid. ¹H NMR (400 MHz, DMSO-d₆) δ 5.90-5.50 (m, 11H), 4.95-4.77 (m, 7H), 4.67 (d, 1H), 4.62-4.42 (m, 7H), 3.83 (bs, 1H), 3.80-3.65 (m, 28H), 3.65-3.30 (m, overlap with water peak).

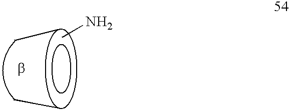

2-Monoazido-β-cyclodextrin 53 (1.00 g, 0.86 mmol) and triphenylphosphine (0.50 g, 1.90 mmol) were dissolved in DMF (20 mL). The reaction mixture was stirred at 90° C. for 18 h, then poured into acetone (1 L). After filtration, the solid was washed with acetone (50 mL) and dissolved in the minimum volume of water. 2-Amino-β-cyclodextrin 54 (0.92 g, 93%) was obtained as a white solid after lyophilisation. ¹H NMR (400 MHz, D₂O) δ 5.00-4.91 (m, 7H), 3.91-3.68 (m, 28H), 3.60-3.42 (m, 28H), 3.26 (t, 1H).

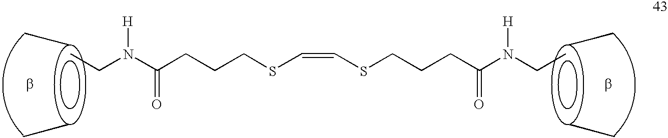

To a solution of 2-amino β-cyclodextrin 54 (0.50 g, 0.44 mmol) in DMF (20 mL) was added 45 (60 mg, 0.22 mmol), 1-hydroxybenzotriazole (HOBT) (89 mg, 0.66 mmol) and 1,3-dicyclohexylcabodiimide (DCC) (0.14 g, 0.66 mmol). The mixture was then heated at 60° C. for 18 h and then poured into acetone (1 L). The resultant precipitate was filtered and purified by reverse-phase column using a H₂O/MeOH solvent gradient (MeOH 20-80%). This gave pure dimer 43 (0.23 g, 42%) as a white solid. ¹H NMR (300 MHz, DMSO-d₆) δ 7.68 (bs, 2H), 6.16 (s, 2H), 5.80-5.67 (m, 28H), 4.81 (m, 14H), 4.47 (m, 14H), 3.62-3.32 (m, 84H), 2.72-2.66 (m, 4H), 2.21-2.10 (m, 4H), 1.84-1.66 (m, 4H); MS (MALDI) m/z (%) 2517 (M+Na⁺, 5).

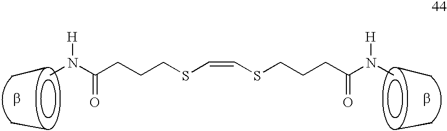

To a solution of 6-amino β-cyclodextrin 50 (0.50 g, 0.44 mmol) in DMF (20 mL) was added 45 (60 mg, 0.22 mmol), 1-hydroxybenzotriazole (HOBT) (89 mg, 0.66 mmol) and 1,3-dicyclohexylcabodiimide (DCC) (0.14 g, 0.66 mmol). The mixture was then heated at 60° C. for 18 h and then poured into acetone (1 L). The resultant precipitate was filtered and purified by reverse-phase column using a H₂O/MeOH solvent gradient (MeOH 20-80%). This yielded pure dimer 44 (0.22 g, 40%) as a white solid. ¹H NMR (300 MHz, DMSO-d₆) δ 7.90 (bs, 2H), 6.20 (s, 2H), 5.99 (d, J=6.1, 2H), 5.79-5.61 (m, 26H), 4.82-4.75 (m, 14H), 4.50-4.45 (m, 14H), 3.62-3.32 (m, 84H), 2.73 (m, 4H), 2.26-2.23 (m, 4H), 1.82-1.79 (m, 4H); MS (MALDI) m/z (%) 2516 (M+Na⁺, 15).

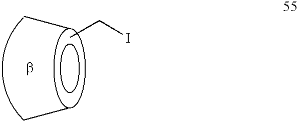

A solution of 6-monotoluenesulfonyl-β-cyclodextrin 48 (20.00 g, 16.8 mmol) and KI (2.00 g, 12.1 mmol) were dissolved in DMF (100 mL). The reaction mixture was stirred under argon at 50° C. for 18 h, and then poured into acetone (1 L). The product was filtered out by frit funnel and washed with acetone, then dissolved in water and lyophilized to give 6-monoiodo-β-cyclodextrin 55 as product (17.05 g, 90%) as a white solid. ¹H NMR (300 MHz, DMSO-d₆) δ 5.83-5.60 (m, 14H), 4.90-4.78 (m, 7H), 4.57-4.40 (m, 6H), 3.94-3.54 (m, 28H).

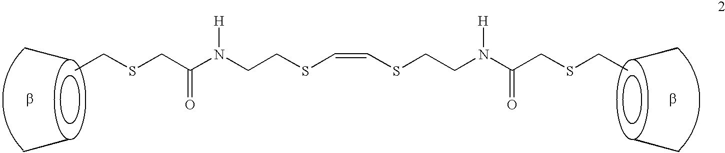

To a solution of diacetate (55 mg, 0.13 mmol) in MeOH (2.5 mL) was added a 25% weight solution of NaOMe in MeOH (0.063 g, 0.29 mmol) and this solution was stirred at 25° C. for 1.25 h. To this solution was then added a solution of 6-monoiodo β-cyclodextrin 55 (0.26 g, 0.21 mmol) in DMF (7 mL) and this solution was heated at 50° C. for 20 h. Upon addition of the cyclodextrin solution the reaction mixture was seen to turn cloudy. This cloudiness had disappeared after 20 h of heating at 50° C. This solution was cooled to 25° C. and concentrated. The resulting solid was purified by reverse-phase column using a H₂O/MeOH solvent gradient (MeOH 0-80%). This gave some pure dimer along with some fractions containing impure material. The relevant impure fractions were collected, concentrated and were further purified by reverse-phase column using a H₂O/MeOH solvent gradient (MeOH 0-60%). Overall, this afforded the desired dimer 2 (94 mg, 35%) as a white solid. TLC R_{f} 0.05 7:7:5 i-PrOH:Ethyl Acetate:Water; ¹H NMR (300 MHz, DMSO-d₆) δ 8.12-.8.08 (m, 2H), 6.22 (s, 2H), 5.80-5.68 (m, 26H), 4.82 (m, 14H), 4.57-4.46 (m, 12H), 3.77-3.33 (m, 84H), 2.85-2.62 (m, 12H); MS (MALDI) m/z (%) 2517 (M+Na⁺, 5).

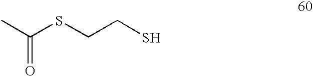

A solution of ethanedithiol 59 (3.50 mL, 41.75 mmol) and acetic anhydride (4.00 mL, 41.75 mmol) in pyridine (10 mL) and CH₂Cl₂ (10 mL) was stirred at 25° C. for 18 h. The solvents were removed under vacuum and the resulting oil distilled under high-vac to yield pure thioacetic acid S-[2-(2-acetylsulfanyl-ethyldisulfanyl)-ethyl]ester 60 (2.20 g, 39%) as a clear liquid. TLC R_{f} 0.42 1:9 Ethyl Acetate:Hexanes; ¹H NMR (300 MHz, CDCl₃) δ 3.13-3.06 (m, 2H), 2.75-2.67 (m, 2H), 2.36 (s, 3H), 1.62 (t, J=8.5, 1H).

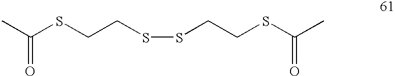

To a solution of mono-acetate 60 (1.03 g, 7.56 mmol) in CH₂Cl₂ (8 mL) and 10% K₂CO₃ in water (8 mL) was slowly added bromine (0.61 g, 0.20 mL, 3.78 mmol). After complete addition the organic layer was separated and the aqueous layer was washed with CH₂Cl₂ (2×15 mL). The combined organic layers were dried (MgSO₄) and concentrated to give thioacetic acid S-[2-(2-acetylsulfanyl-ethyldisulfanyl)-ethyl] ester 61 (0.90 g, 88%) as a yellow oil. TLC R_{f} 0.20 1:9 Ethyl Acetate:Hexanes; ¹H NMR (300 MHz, CDCl₃) δ 3.24-3.19 (m, 4H) 2.90-2.84 (m, 4H), 2.36 (s, 6H); MS (FAB) m/z (%) 271 (M+H+, 30), 270 (M+H⁺, 20).

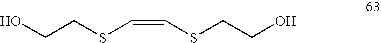

A solution of 2-mercaptoethanol 64 (11.14 g, 10.00 mL, 0.14 mol) and NaOH (5.79 g, 0.145 mol) in EtOH (40 mL) was stirred at 0° C. for 30 min. To this solution was added dropwise a solution of cis-1,2-dichloroethylene (6.91 g, 5.48 mL, 0.07 mol) in EtOH (5 mL). This solution was heated at 80° C. for 18 h. This mixture was cooled to 25° C., diluted with water (100 mL) and washed with diethyl ether (3×50 mL) The combined organic layers were washed with water (2×75 mL), dried (MgSO₄) and concentrated to give the crude product. This was purified by column chromatography on silica (75% ethyl acetate/hexanes-ethyl acetate) to give pure 2-[2-(2-hydroxy-ethylzulfanyl)-vinylsulfanyl]ethanol 63 (9.10 g, 72%) as a light yellow liquid. IR (ν) 3334, 2920, 2866, 1544, 1409, 1283, 1046, 1011, 841, 638; ¹H NMR (300 MHz, CDCl₃) δ 6.17 (s, 2H), 3.78 (ap. q, J=6.0, 4H), 2.91 (t, J=5.4, 4H), 2.34 (bt, J=5.4, 2H); ¹³C NMR (75 MHz, CDCl₃) δ 124.9, 61.4, 37.4; MS (FAB) m/z (%) 180 (M⁺, 100).

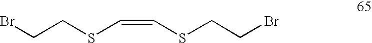

To a 0° C. solution of triphenylphosphine (6.55 g, 24.96 mmol) in CH₂Cl₂ (15 mL) was carefully added bromine (2.93 g, 0.94 mL, 18.3 mmol). This was warmed to 25° C. and diluted with CH₂Cl₂ (60 mL). To this cloudy solution was added a solution of 63 (1.50 g, 8.32 mmol) in CH₂Cl₂ (8 mL)₁ during which time the solution became clear. This mixture was stirred at 25° C. for 2 h. The mixture was then concentrated to give a white solid. This solid was washed with hexanes (20 mL) and diethyl ether (3×20 mL). These were combined and concentrated to give 1,2-bis(2-bromo-ethylsulfanyl)-ethene 65 as an ˜1:1 mixture with triphenyl phosphine oxide (yield of dibromide=2.40 g, 95%). IR (ν) 3053, 3010, 1589, 1546, 1476, 1432, 1195, 1120, 747, 721, 664, 619; ¹H NMR (300 MHz, CDCl₃) δ 6.14 (s, 2H), 3.47 (t, J=7.8, 4H) 3.10 (t, J=7.8, 4H); ¹³C NMR (75 MHz, CDCl₃) δ 124.4, 35.9, 30.2; MS (FAB) m/z (%) 305 (M+H⁺, 10).

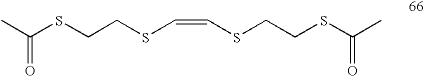

To a solution of dibromide 65/triphenyl phosphine oxide ˜1:1 mixture (1.20 g of dibromide, 3.92 mmol) in DMF (15 mL) was added potassium thioacetate (2.24 g, 19.6 mmol) and this mixture was heated to 80° C. for 24 h. The resulting dark mixture was then cooled to 25° C. poured into water (150 mL) and extracted with diethyl ether (4×50 mL). The combined organic layers were dried (MgSO₄) and concentrated to give the crude product. This was purified by column chromatography on silica (15% ethyl acetate/hexanes) to give pure thioacetic acid S-{2-[2-(2-acetylsulfanyl-ethylsulfanyl0-vinylsulfanyl]ethyl}ester 66 (0.54 g, 47%) as an off white solid. TLC R_{f} 0.28 15:85 Ethyl Acetate:Hexanes; Mp 58° C.; ¹H NMR (300 MHz, CDCl₃) δ 65.24 (s, 2H), 3.12-3.07 (M, 4H), 2.90-2.84 (m, 4H), 2.35 (s, 6H)); ¹³C NMR (75 MHz, CDCl₃) δ 195.3, 124.2, 33.8, 30.7, 29.9; MS (APCI) m/z (%) 295 (M−H⁺, 100) 270 (M+H⁺)

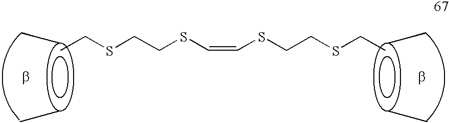

To a solution of diacetate 66 (0.15 g, 0.51 mmol) in MeOH (9 mL) was added 25% weight solution of NaOMe in MeOH (0.24 g, 0.26 mL, 1.11 mmol) and this solution was stirred at 25° C. for 2 h. To this solution was added a solution of 6-monolodo β-cyclodextrin 55 (1.00 g, 0.80 mmol) in DMF (30 mL) and this solution was heated at 50° C. for 20 h. Upon addition of the cyclodextrin solution the reaction mixture was seen to turn cloudy. This cloudiness had disappeared after 20 h of heating at 50° C. This solution was cooled to 25° C. and concentrated under vacuum. The resulting solid was purified by reverse-phase column using a H₂O/MeOH solvent gradient (MeOH 0-80%). This gave some pure dimer along with some fractions containing impure material. The relevant impure fractions were collected, concentrated and were further purified by reverse-phase column using a H₂O/MeOH solvent gradient (MeOH 0-60%). The mixed fractions appeared to be contaminated with of 6-monoiodo β-cyclodextrin 55 and in an attempt to remove this the concentrated mixed fractions were dissolved in 10% NaOH aqueous solution (10 mL) and heated to 60° C. for 3 days. The solution was then cooled to 25° C., pH adjusted to 7 using dilute HCl and filtered. The resulting solution was purified by reverse-phase column using a H₂O/MeOH solvent gradient (MeOH 0-50%). Overall, this afforded the desired dimer 67 (0.35 g, 36%) as a white solid. TLC R_{f} 0.13 7:7:5 i-PrOH:Ethyl Acetate:Water; ¹H NMR (300 MHz, DMSO-d₆) δ 6.24 (s, 2H), 5.78-5.67 (m, 28H), 4.84 (s, 14H), 4.49-4.43 (m, 12H), 3.87-3.30 (m, 80H), 3.05-2.64 (m, 12H).

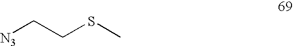

To a solution of NaN₃ (3.51 g; 54.0 mmol; 3.0 eq.) in H₂O (20 mL) was added a solution of 2-chloroethyl methyl sulfide 68 (2.0 g; 18.0 mmol; 1.0 eq.) in CH₂Cl₂ (20 mL) and a catalytic amount (˜10 mol %) of tetrabutylammonium chloride. This mixture was rigorously stirred at 30° C. for 3 h. The organic layer was separated from the aqueous layer and was dried (MgSO₄), filtered and concentrated to yield 1-azido-2-methylsulfanyl-ethane 69 (1.70 g, 81%) as a clear oil. ¹H NMR (300 MHz, CDCl₃) δ 3.48 (t, J=7.0, 2H), 2.70 (t, J=7.0, 2H), 2.17 (s, 3H).

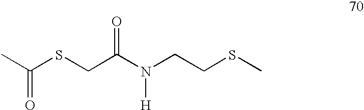

To a solution of 1-azido-2-methylsulfanyl-ethane 69 (1.00 g; 8.6 mmol; 1.0 eq.) and triphenylphosphine (2.24 g; 8.6 mmol; 1.0 eq.) in THF (30 mL), was added water (0.15 mL; 8.6 mmol; 1.0 eq.). The reaction mixture was heated to 35° C. for 3 h, after which it was cooled to 25° C. and then to 0° C. To this mixture was added freshly distilled triethylamine (1.8 mL; 12.9 mmol; 1.5 eq.). Chloroacetyl chloride (1.46 g; 12.9 mmol; 1.5 eq.) was then added dropwise, and the resulting reaction mixture was allowed to warm to 25° C. Potassium thioacetate (4.90 g; 43 mmol; 5.0 eq.) was then added to the reaction mixture, which was then heated to 50° C. for 18 h. The solution was then concentrated under reduced pressure, and the residue was purified by column chromatography on silica (gradient: 100% CH₂Cl₂ to 10% MeOH/CH₂Cl₂) to give thioacetic acid S-[(methylsulfanyl-ethyl carbamoyl)methyl]ester 70 (1.20 g, 67%) as an off-white solid. Rf=0.85 (10% MeOH/CH₂Cl₂); ¹H NMR (400 MHz, CDCl₃) δ 6.68 (bs, 1H), 3.56 (s, 2H), 3.44 (m, 2H), 2.62 (t, J=6.5, 2H), 2.41 (s, 3H), 2.10 (s, 3H).

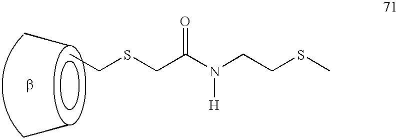

To a solution of 70 (0.50 g; 2.41 mmol; 30.0 eq.) in MeOH (50 mL) was added NaOH (0.20 g; 5.0 mmol; 62.2 eq.). The resulting mixture was stirred at 50° C. for 10 minutes. TLC showed the disappearance of the starting thioacetate and an appearance of a new spot at Rf=0.65 (10% MeOH/CH₂Cl₂). The solution was concentrated under reduced pressure. To this residue was added a mixture of β-CD-6-I (0.10 g; 0.08 mmol; 1.0 eq.) and K₂CO₃ (55 mg; 0.4 mmol; 5.0 e.q.) in DMF (20 mL). The reaction flask was evacuated and backfilled with argon three times. The mixture was heated to 55° C. for 24 h. Water (180 mL) was then added to the reaction mixture. This mixture was filtered and was then purified by reverse phase column chromatography eluted with MeOH/H₂O mixture (linear gradient 80% H₂O—80% MeOH). The methanol of the fractions that contained the product was removed under reduced pressure, and the residual aqueous solution was lyophilized. This gave monomer 71 (80 mg, 78%) as a white solid. Rf=0.56 (7:7:7:4 iPrOH:EtOAc:H₂O:NH₄OH); ¹H NMR (300 MHz, D₂O) δ 5.05-4.90 (m, 7H), 4.10-3.17 (m, 44H), 2.85 (m, 2H) 2.59 (t, J=6.6, 2H), 2.14 (s, 3H).

### REFERENCES

Adam, W. and Cliento, G. (1983) Angew. Chem., Int. Ed. Engl. 22: 529-542.

Ali, H., van Lier, J. E. (1999) Chem. Rev. 99: 2379-2450.

Bartlett, P. D. (1976) Chem. Soc. Rev. 79: 149-163.

Breslow, R., Greenspoon, N., Guo, T., Zarzycki, R. (1989) J. Am. Chem. Soc. 111: 8296-8297.

Breslow, R., Ruebner, A., Yang, Z. A HYDROPHILIC CARRIER FOR PHOTOSENSITIZERS THAT CLEAVES WHEN THEY CATALYZE THE FORMATION OF SINGLET OXYGEN, U.S. Ser. No. 09/352,529, filed Jul. 13, 1999, now allowed.

Clennan, E. L. and Nagraba, K. (1988) J. Am. Chem. Soc. 110: 4312-4318.

Dougherty, T. J., Gomer, C. J., Henderson, B. W., Jori, G., Kessel, D., Korbelik, M., Moan, J., Peng, Q. (1998) Photodynamic therapy. J. Natl. Cancer Inst. 90: 889-905.

Ellis, G., Romney-Alexander, T. (1987) Chem. Rev. 87: 779-794.

Foote, C. S. (1971) Pure Appl. Chem. 27: 635-645.

Frimer, A. A. (1979) Chem. Rev. 79: 359-387.

Kearns, D. R. (1971) Chem. Rev. 71: 395-427.

Kliesch, H., A. Weitemeyer, S. Muller, and D. Wöhrle (1995) Liebigs Ann 1269-1273.

Kohn, M. (1951) J. Am. Chem. Soc. 73: 480.

Lawrence, D. S., Whitten, D. G. (1996) Photochemistry and Photobiology 64(6): 923-935.

Leznoff, C. C. (1989) Syntheses of Metal-Free Substituted Phthalocyanines, In: Leznoff, C. C. and Lever, A. B. P., Eds., VCH: New York, Vol. 1, pp 6-20.

Marcuccio, S. M., Svirskaya, P. I., Greenberg, S., Lever, A. B. P., Leznoff, C. C., Tomer, K. B. (1985) Can. J. Chem. 63: 3057-3069.

Metz, J., Schneider, O, Hanack, M. (1984) Inorg. Chem. 23: 1065-1071.

Meyer, W. L., Brannon, M. J., Burgos, C. G., Goodwin, T. E., Howard, R. W. (1985) J. Org. Chem. 50: 438-447.

Mitchell, M., Lai, Y. H., Williams, R. V. (1979) J. Am. Chem. Soc. 101: 4733-4735.

Moser, J. G., A. Heuermann, P. Oehr, H. Scheer, A. Vervoorts, and S. Andrees (1994) SIPE. Conf. Proc. 92: 2325.

Rekharsky, M. V., Inoue, Y. (1998) Chem. Rev. 98: 1875-1917.

Ruebner, A., D. Kirsch, S. Andrees, W. Decker, B. Roeder, B. Spengler, R. Kaufmann, and J. G. Moser (1997) Dimeric cyclodextrin carriers with high binding affinity to porphyrinoid photosensitizers. Journal of Inclusion Phenomena and Molecular Recognition in Chemistry 27: 69-84.

Ruebner, A., J. G. Moser, D. Kirsch, B. Spengler, S. Andrees, and S. Roehrs (1996) Synthesis of β-cyclodextrin dimers as carrier systems for photodynamic therapy of cancer. In: Szejtli, J. and L. Szente (Eds.), Proceedings of the Eight International Symposium on Cyclodextrons, Kluwer Academic Publishers, pp 77-80.

Ruebner, A., Yang, Z., Leung, D., Breslow, R. (1999) A cyclodextrin dimer with a photocleavable linker as a possible carrier for the photosensitizer in photodynamic tumor therapy. Proceedings of the National Academy of Science 96(26): 14692-14693.

Schaap, A. P. and Zaklika, K. A. (1979) in Wasserman, H. H.; Murray, R. W. Singlet Oxygen; Academic Press: New York, pp 173-242.

Sternberg, E. D., Dolphin, D., Bruckner, C. (1998) Porpyhrin-based photosensitizers for use in photodynamic therapy. Tetrahedron 54: 4151-4202.

Takakis, I. M., Hadjimihalakis, P. M., Tsantali, G. G., Pilini, H. (1992) J. Heterocyclic Chem. 29: 123-128.

Yang, Z., Breslow, R. (1997) Tetrahedron Lett. 38: 6171-6172.

Yuan, D., Dong, S. D., Breslow, R. (1998) Tetrahedron Lett. 62: 847-855.

What is claimed is:

1. A composition of matter comprising two β-cyclodextrin molecules and a cleavable linker joining each such β-cyclodextrin, wherein the cleavable linker comprises a carbon-carbon double bond substituted on both ends, wherein the cleavable linker is cleavable by singlet oxygen, and wherein the composition of matter is selected from the group consisting of: 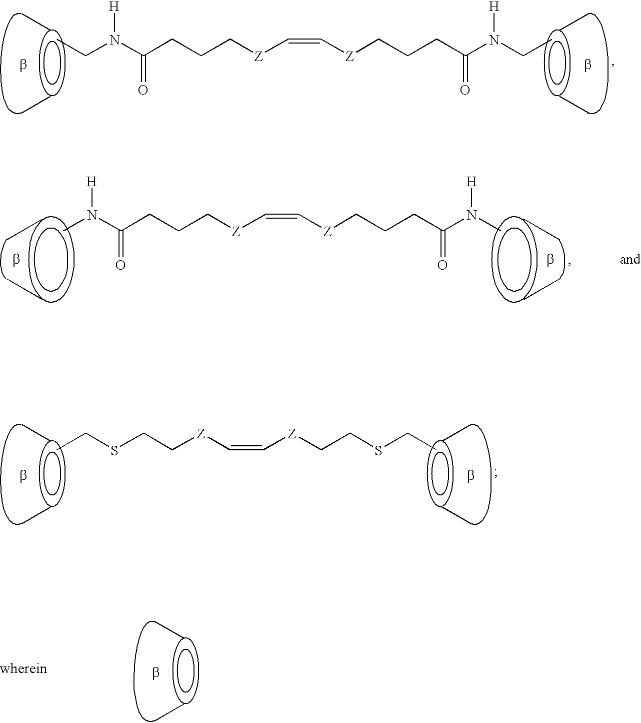
=beta-cyclodextrin; and
wherein Z is C₁-C₄ alkyl, NH, N(C₁-C₄ alkyl) O, or S.

2. The composition of matter of claim 1, wherein Z is S.

3. The composition of matter of claim 2, having the structure:

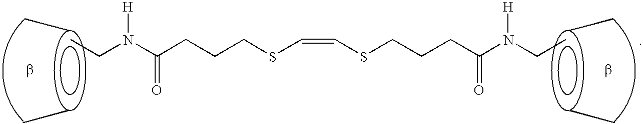

4. The composition of matter of claim 2, having the structure:

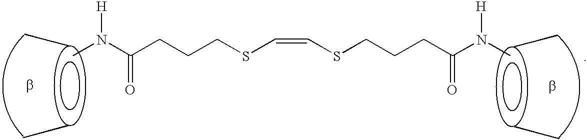

5. The composition of matter of claim 2, having the structure:

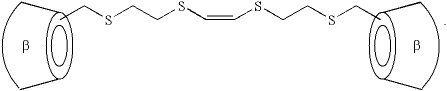

6. A composition which comprises a hydrophilic matrix comprising the composition of matter of any of claim 1, 2, 3, 4 or 5, and a photosensitizer encapsulated within the matrix.

7. The composition of claim 6, wherein the photosensitizer is a porphyrin, a phthalocyanine, a naphthalocyanine, a chlorin, a pheophorbide, or a bacteriopheophorbide.

8. The composition of claim 7, wherein the photosensitizer is a phthalocyanine.

9. The composition of claim 8, wherein the phthalocyanine has the structure: 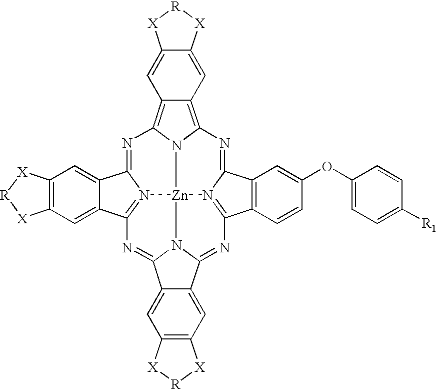
wherein X is C₁-C₄ alkyl, NH, N(C₁-C₄ alkyl), O, or S;
wherein R₁ is —CO₂H, —CO₂⁻, —N⁺(CH₃)₃, —SO₃H, or —SO₃⁻; and
wherein R is 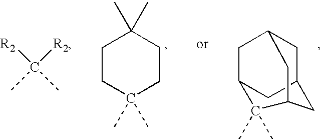
where the dashed lines indicate the attachments to X, and where R₂ is C₁-C₃ alkyl.

10. The composition of claim 9, wherein X is O, and R₁ is —SO₃H.

11. The composition of claim 10, wherein the phthalocyanine has the structure:

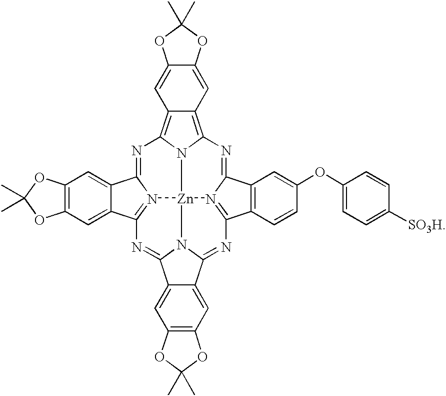

12. The composition of claim 10, wherein the phthalocyanine has the structure:

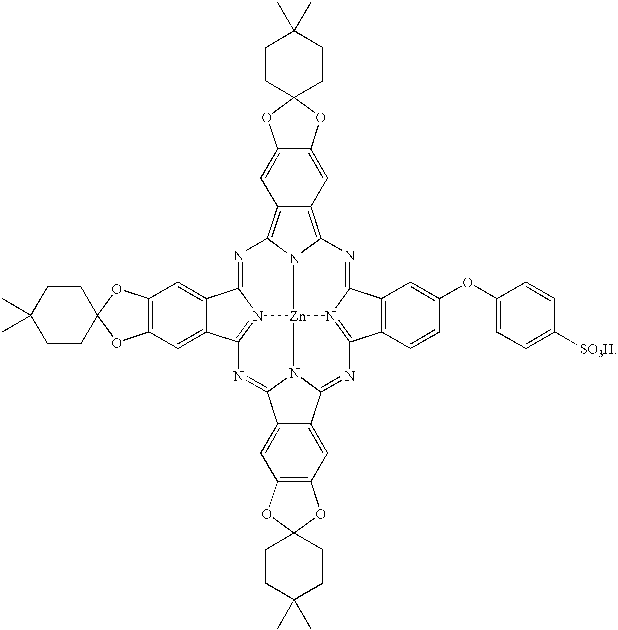

13. The composition of claim 10, wherein the phthalocyanine has the structure:

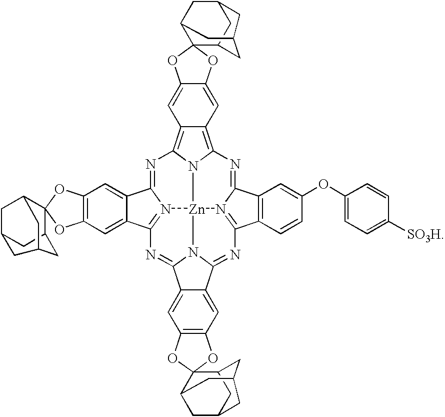

14. A compound having the structure: 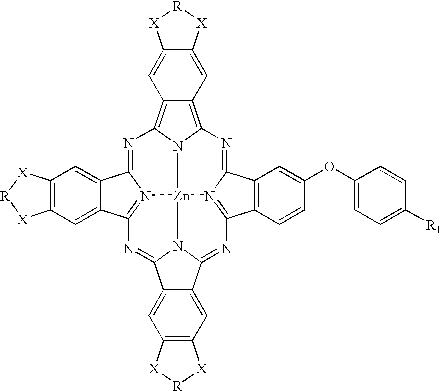
wherein X is C₁-C₄ alkyl, NH, N(C₁-C₄ alkyl), O, or S;
wherein R₁ is —CO₂H, —CO₂⁻, —N⁺(CH₃)₃, —SO₃H, or —SO₃³; and
wherein R is 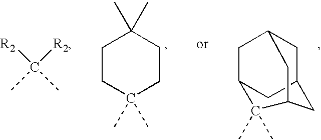
where the dashed lines indicate the attachments to X, and where R₂ is C₁-C₃ alkyl.

15. The compound of claim 14, wherein X is O, and R₁ is —SO₃H.

16. The compound of claim 15 having the structure:

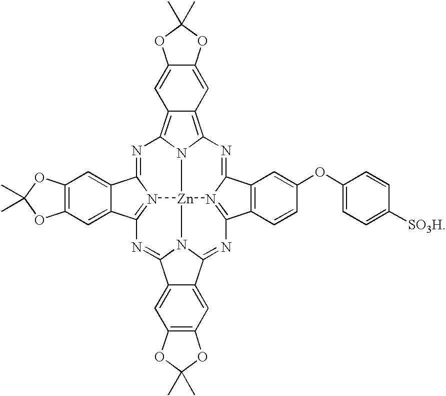

17. The compound of claim 15 having the structure:

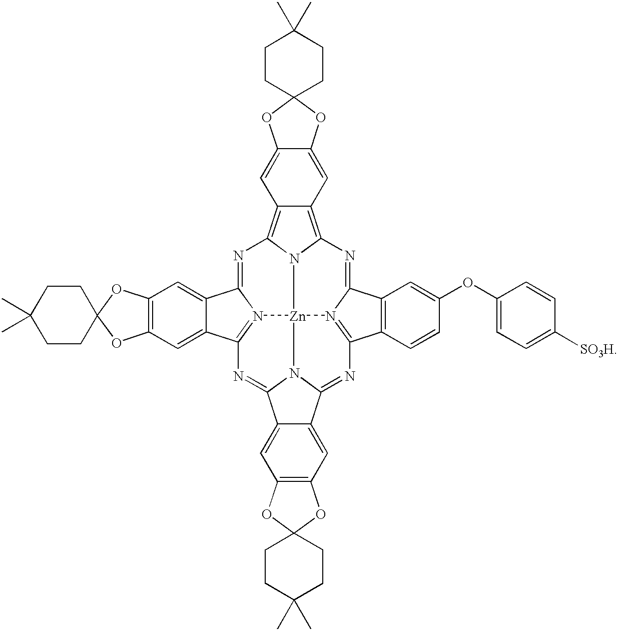

18. The compound of claim 15 having the structure:

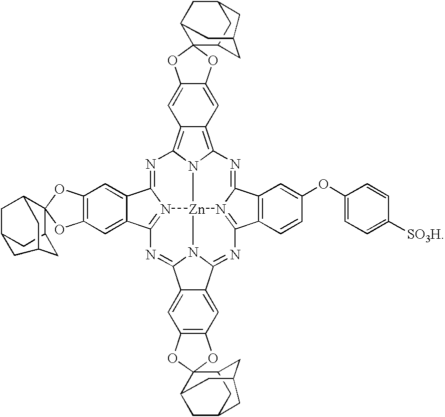

19. A composition which comprises a hydrophilic matrix comprising:
i) the compound of any of claim 14, 15, 16, 17, or 18 encapsulated within the matrix, and
ii) a composition of matter comprising two β-cyclodextrin molecules and a cleavable linker joining each such β-cyclodextrin, wherein the cleavable linker comprises a carbon-carbon double bond substituted on one or both ends by an electron rich atom, and the cleavable linker is cleavable by singlet oxygen.

20. The composition of claim 19, wherein the electron rich atom is sulfur, oxygen, or nitrogen.

21. The composition of claim 19, wherein the composition of matter is selected from the group consisting of: 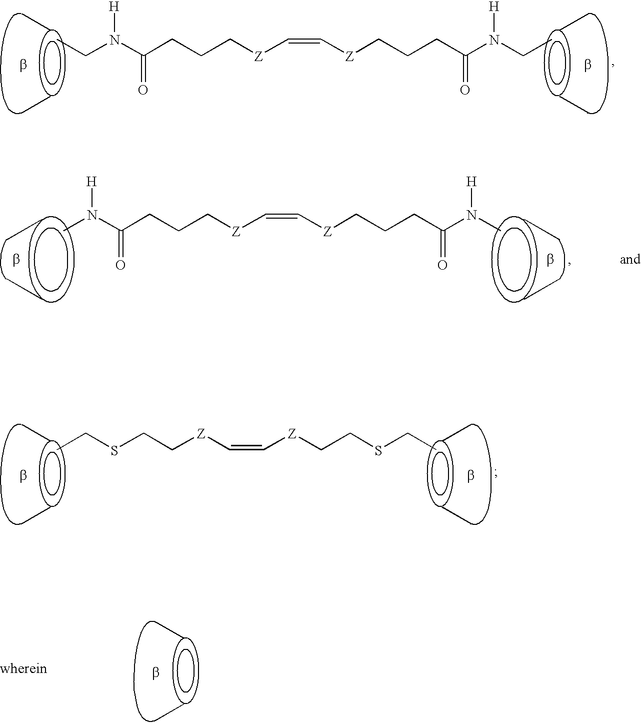
=beta-cyclodextrin; and
wherein Z is C₁-C₄ alkyl, NH, N(C₁-C₄ alkyl), O, or S.

22. The composition of claim 21, wherein Z is S.

23. The composition of claim 22, wherein the composition of matter has the structure:

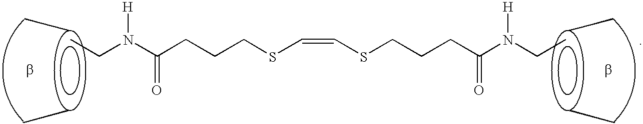

24. The composition of claim 22, wherein the composition of matter has the structure:

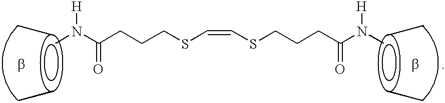

25. The composition of claim 22, wherein the composition of matter has the structure:

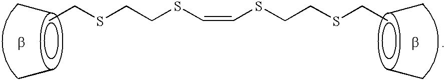

26. The composition of claim 6 or 19, wherein the cleavable linker is cleavable upon exposure to light of a wavelength appropriate for absorption by the photosensitizer.

27. The composition of claim 6 or 19, wherein the photosensitizer is released when the cleavable linker is cleaved.

28. A method of killing a tumor cell which comprises contacting the tumor cell with the composition of claim 26 and exposing the composition to light so as to cleave the cleavable linker and release the photosensitizer, wherein absorption of light by the photosensitizer excites the photosensitizer and the tumor cell is killed by singlet oxygen that is formed by energy transfer from the excited photosensitizer.

29. A method of killing a tumor cell in a subject which comprises:
(a) administering the composition of claim 26 to the subject;
(b) directing light at the tumor cell so as to expose the composition to light and cleave the cleavable linker thereby releasing the photosensitizer, wherein absorption of light by the photosensitizer excites the photosensitizer and generates singlet oxygen that is formed by energy transfer from the excited photosensitizer;
(c) allowing additional composition to diffuse to the tumor cell; and
(d) repeating steps (b) and (c) until sufficient singlet oxygen is generated to kill the tumor cell.

30. The method of claim 29, wherein the photosensitizer is concentrated at the tumor cell.

31. The method of claim 28, wherein a plurality of converging light beams is used to focus light on the tumor cell.

32. The method of claim 29, wherein a plurality of converging light beams is used to focus light on the tumor cell.
